SEQUENCE LISTING (62 sequences)

SEQ ID NO: 1 - DNA, 22 bp, artificial
  hsa-miR-15b
uagcagcacaucaugguuuaca

SEQ ID NO: 2 - DNA, 21 bp, artificial
  hsa-miR-23a
aucacauugccagggauuucc

SEQ ID NO: 3 - DNA, 22 bp, artificial
  hsa-miR-146b
ugagaacugaauuccauaggcu

SEQ ID NO: 4 - DNA, 21 bp, artificial
  hsa-miR-23b
aucacauugccagggauuacc

SEQ ID NO: 5 - DNA, 22 bp, artificial
  hsa-miR-130a
cagugcaauguuaaaagggcau

SEQ ID NO: 6 - DNA, 22 bp, artificial
  hsa-miR-199a
acaguagucugcacauugguua

SEQ ID NO: 7 - DNA, 23 bp, artificial
  hsa-miR-191
caacggaaucccaaaagcagcug

SEQ ID NO: 8 - DNA, 22 bp, artificial
  hsa-miR-25*
cauugcacuugucucggucuga

SEQ ID NO: 9 - DNA, 22 bp, artificial
  hsa-miR-1
uggaauguaaagaaguauguau

SEQ ID NO: 10 - DNA, 23 bp, artificial
  hsa-miR-9
ucuuugguuaucuagcuguauga

SEQ ID NO: 11 - DNA, 23 bp, artificial
  hsa-miR-10a
uacccuguagauccgaauuugug

SEQ ID NO: 12 - DNA, 21 bp, artificial
  hsa-miR-629
uggguuuacguugggagaacu

SEQ ID NO: 13 - DNA, 22 bp, artificial
  hsa-miR-1262
augggugaauuuguagaaggau

SEQ ID NO: 14 - DNA, 22 bp, artificial
  hsa-miR-452
aacuguuugcagaggaaacuga

SEQ ID NO: 15 - DNA, 18 bp, artificial
  hsa-miR-1827
ugaggcaguagauugaau

SEQ ID NO: 16 - DNA, 22 bp, artificial
  hsa-miR-628-5p
augcugacauauuuacuagagg

SEQ ID NO: 17 - DNA, 23 bp, artificial
  hsa-miR-181a-2*
aacauucaacgcugucggugagu

SEQ ID NO: 18 - DNA, 22 bp, artificial
  miR-127-3p
ucggauccgucugagcuuggcu

SEQ ID NO: 19 - DNA, 22 bp, artificial
  hsa-miR-433
aucaugaugggcuccucggugu

SEQ ID NO: 20 - DNA, 21 bp, artificial
  hsa-miR-219
ugauuguccaaacgcaauucu

SEQ ID NO: 21 - DNA, 23 bp, artificial
  hsa-miR-886-5p
cgggucggaguuagcucaagcgg

SEQ ID NO: 22 - DNA, 22 bp, artificial
  hsa-miR-21*
uagcuuaucagacugauguuga

SEQ ID NO: 23 - DNA, 22 bp, artificial
  hsa-miR-21
uagcuuaucagacugauguuga

SEQ ID NO: 24 - DNA, 21 bp, artificial
  hsa-miR-142-5p
cauaaaguagaaagcacuacu

SEQ ID NO: 25 - DNA, 22 bp, artificial
  hsa-miR-92b*
uauugcacucgucccggccucc

SEQ ID NO: 26 - DNA, 22 bp, artificial
  hsa-miR-15a
uagcagcacauaaugguuugug

SEQ ID NO: 27 - DNA, 21 bp, artificial
  hsa-miR-106b
uaaagugcugacagugcagau

SEQ ID NO: 28 - DNA, 21 bp, artificial
  hsa-miR-27a
uucacaguggcuaaguuccgc

SEQ ID NO: 29 - DNA, 22 bp, artificial
  hsa-miR-365
uaaugccccuaaaaauccuuau

SEQ ID NO: 30 - DNA, 21 bp, artificial
  hsa-miR-143*
ugagaugaagcacuguagcuc

SEQ ID NO: 31 - DNA, 22 bp, artificial
  hsa-miR-186
caaagaauucuccuuuugggcu

SEQ ID NO: 32 - DNA, 22 bp, artificial
  hsa-miR-34a
uggcagugucuuagcugguugu

SEQ ID NO: 33 - DNA, 22 bp, artificial
  hsa-miR-199b-3p
acaguagucugcacauugguua

SEQ ID NO: 34 - DNA, 17 bp, artificial
  hsa-miR-1274b
ucccuguucgggcgcca

SEQ ID NO: 35 - DNA, 23 bp, artificial
  hsa-miR-34c-5p
aggcaguguaguuagcugauugc

SEQ ID NO: 36 - DNA, 22 bp, artificial
  hsa-miR-223*
ugucaguuugucaaauacccca

SEQ ID NO: 37 - DNA, 22 bp, artificial
  hsa-miR-204
uucccuuugucauccuaugccu

SEQ ID NO: 38 - DNA, 22 bp, artificial
  hsa-miR-99b*
cacccguagaaccgaccuugcg

SEQ ID NO: 39 - DNA, 21 bp, artificial
  hsa-miR-31
aggcaagaugcuggcauagcu

SEQ ID NO: 40 - DNA, 22 bp, artificial
  hsa-miR-532-5p
caugccuugaguguaggaccgu

SEQ ID NO: 41 - DNA, 18 bp, artificial
  hsa-miR-1308
gcaugggugguucagugg

SEQ ID NO: 42 - DNA, 22 bp, artificial
  hsa-miR-210
cugugcgugugacagcggcuga

SEQ ID NO: 43 - DNA, 22 bp, artificial
  hsa-miR-16
uagcagcacguaaauauuggcg

SEQ ID NO: 44 - DNA, 22 bp, artificial
  hsa-miR-126
ucguaccgugaguaauaaugcg

SEQ ID NO: 45 - DNA, 22 bp, artificial
  hsa-miR-24
uggcucaguucagcaggaacag

SEQ ID NO: 46 - DNA, 22 bp, artificial
  hsa-miR-598
uacgucaucguugucaucguca

SEQ ID NO: 47 - DNA, 22 bp, artificial
  hsa-miR-374b
auauaauacaaccugcuaagug

SEQ ID NO: 48 - DNA, 22 bp, artificial
  hsa-miR-184
uggacggagaacugauaagggu

SEQ ID NO: 49 - DNA, 23 bp, artificial
  hsa-miR-330-3p
gcaaagcacacggccugcagaga

SEQ ID NO: 50 - DNA, 23 bp, artificial
  hsa-miR-9*
ucuuugguuaucuagcuguauga

SEQ ID NO: 51 - DNA, 20 bp, artificial
  hsa-miR-760
cggcucugggucugugggga

SEQ ID NO: 52 - DNA, 21 bp, artificial
  hsa-miR-379
ugguagacuauggaacguagg

SEQ ID NO: 53 - DNA, 23 bp, artificial
  hsa-miR-7
uggaagacuagugauuuuguugu

SEQ ID NO: 54 - DNA, 21 bp, artificial
  hsa-miR-128
ucacagugaaccggucucuuu

SEQ ID NO: 55 - DNA, 22 bp, artificial
  hsa-miR-543
aaacauucgcggugcacuucuu

SEQ ID NO: 56 - DNA, 22 bp, artificial
  hsa-miR-485-5p
agaggcuggccgugaugaauuc

SEQ ID NO: 57 - DNA, 21 bp, artificial
  hsa-miR-499-5p
uuaagacuugcagugauguuu

SEQ ID NO: 58 - DNA, 22 bp, artificial
  hsa-miR-129-3p
aagcccuuaccccaaaaagcau

SEQ ID NO: 59 - DNA, 20 bp, artificial
  hsa-miR-124
uaaggcacgcggugaaugcc

SEQ ID NO: 60 - DNA, 22 bp, artificial
  hsa-miR-219-2-3p
agaauuguggcuggacaucugu

SEQ ID NO: 61 - DNA, 42 bp, artificial
  sequence 61
cgcaaggaugacacgcaaauucgugaagcguuccauauuuuu

SEQ ID NO: 62 - DNA, 22 bp, artificial
  sequence 62
aucaugaugggcuccucggugu

(19) United States
(12) Patent Application Publication
(10) Pub. No.: US 2011/0301221 A1
(43) Pub. Date: Dec. 8, 2011

(54) DIAGNOSIS, PROGNOSIS AND TREATMENT OF GLIOBLASTOMA MULTIFORME

(75) Inventors: Biaoyang Lin, Bothell, WA (US); Gregory Foltz, E Seattle, WA (US)

(73) Assignee: SWEDISH HEALTH SERVICES, Seattle, WA (US)

(21) Appl. No.: 13/123,711

(22) PCT Filed: Oct. 9, 2009
(86) PCT No.: PCT/US09/60185

§ 371 Date: Aug. 3, 2011

Related U.S. Application Data
(60) Provisional application No. 61/104,659, filed on Oct. 10, 2008.

(51) Int. Cl. A61K 31/7105; C12Q 1/68; A61P 35/00; C40B 30/04

(52) U.S. Cl. 514/44 A; 506/9; 435/6.12

(57) ABSTRACT

The present invention in one aspect relates generally to the identification, provision and use of a plurality of biomarkers to provide risk assessment of a subject having glioblastoma multiforme, and products and processes related thereto. In one aspect, a novel plurality of biomarkers as described herein is provided to determine a risk of glioblastoma multiforme. In another aspect, a novel plurality of biomarkers as described herein is provided to diagnose a subject having glioblastoma multiforme. In yet another aspect are methods for treating a subject having glioblastoma multiforme by administering one or more therapeutic regimens for glioblastoma multiforme. In yet another aspect are nucleic acid arrays comprising nucleic acid probes that hybridize to one or more glioblastoma multiforme genes.

### CROSS-REFERENCE

This application claims the benefit of U.S. Provisional Application No. 61/104,659, filed Oct. 10, 2008, which application is incorporated herein by reference in its entirety.

### BACKGROUND OF THE INVENTION

On a worldwide basis, tumors of the brain belong to the widespread tumors observed for young people. In the United States, 180,000 new cases of different brain tumors are found to occur, in particular tumors of the heterogeneous group of malign gliomas, which covers anaplastic astrocytomes, glioblastomes and, especially, the highly malign glioblastoma multiforme (GBM). The fact that preparations for pharmacologically suppressing the formation of recidives are not yet available, results into a high mortality of young people affected by such tumors.

Brain tumors may be a target for clinical gene therapy because, inter alia, the disease is rapidly fatal and no effective therapies are available [Rainov, N. G., A phase III clinical evaluation of herpes simplex virus type 1 thymidine kinase and ganciclovir gene therapy as an adjuvant to surgical resection and radiation in adults with previously untreated glioblastoma multiforme, Hum Gene Ther, 11:2389-401 (2000); Lang, F. F. et al., Phase I trial of adenovirus-mediated p53 gene therapy for recurrent glioma: biological and clinical results, J Clin Oncol, 21:2508-18 (2003); Sandmair, A. M. et al., Thymidine kinase gene therapy for human malignant glioma, using replication-deficient retroviruses or adenoviruses, Hum Gene Ther, 11:2197-205 (2000); Klatzmann, D. et al., A phase I/II study of herpes simplex virus type 1 thymidine kinase “suicide” gene therapy for recurrent glioblastoma, Study Group on Gene Therapy for Glioblastorna, Hum Gene Ther, 9:2595-604 (1998)].

GBM is an aggressive, primary tumor of the central nervous system (Furnari F B, et al. Genes Dev. 2007; 21: 2683-7270). Because of their intrinsic, infiltrative nature, GBMs follow a malignant clinical course. Classified as World Health Organization grade IV astrocytic tumors, GBMs have a pronounced mitotic activity, substantial tendency toward neoangiogenesis (microvascular proliferation), necrosis, and proliferative rates three to five times higher than grade III tumors, the anaplastic astrocytomas. The clinical behavior of GBMs is often mimicked by unusual pathological presentations, which gave rise to the old moniker of “glioblastoma multiforme.” Even with the survival advantage provided by the recently developed protocol of concurrent chemoradiation followed by adjuvant alkylating chemotherapy with temozolomide (the Stupp regimen), the prognosis of patients with GBM remains poor, with median overall survival in the range of 9-15 months and two-year survival rates of 26% in the most favorable subgroup (Stupp R, et al. N Engl J Med. 2005; 352: 987-996). The outlook for patients with malignant gliomas is poor. Median survival for patients with moderately severe (grade III) malignant gliomas is three to five years. For patients with the most severe, aggressive form of malignant glioma (grade IV glioma or glioblastoma multiforme), median survival is less than a year.

### SUMMARY OF THE INVENTION

Provided herein is a method of diagnosing whether a subject has, or is at risk for developing, a brain tumor or related disease, comprising: (i) obtaining from a biological sample from the subject a set of expression profiles of a plurality of brain tumor marker genes from Table 1, (ii) comparing (a) a set of expression profiles of brain tumor marker genes in a biological sample from the subject, the set comprising expression profiles of a plurality of brain tumor marker genes from Table 1, to (b) a set of expression profiles of brain tumor marker genes in a biological sample from a control subject; and (iii) providing a diagnosis for, or a risk assessment of, a brain tumor or related disease based on the comparison. In one embodiment, the method further comprises obtaining the set of expression profiles prior to the comparing step.

Provided herein is a method of diagnosing whether a subject has, or is at risk for developing, a brain tumor or related disease, comprising: (i) comparing (a) a set of expression profiles of brain tumor marker genes in a biological sample from the subject, the set comprising expression profiles of a plurality of brain tumor marker genes from Table 2, to (b) a set of expression profiles of brain tumor marker genes in a biological sample from a control subject; and (ii) providing a diagnosis for, or a risk assessment of, a brain tumor or related disease based on the comparison. In one embodiment, the method further comprises obtaining the set of expression profiles prior to the comparing step.

Provided herein is a method for identifying a subject having a brain tumor, comprising determining expression profiles of no more than five to five hundred genes in a biological sample comprising cells from a subject, wherein at least 20% of the genes are selected from the brain tumor marker genes listed in Table 1.

Provided herein is a method for identifying a subject having a brain tumor or related disease, comprising determining expression profiles of no more than five to five hundred genes in a biological sample comprising cells from a subject, wherein at least 20% of the genes are selected from the brain tumor marker genes listed in Table 2.

Provided herein is a method of determining the prognosis of a subject with a brain tumor or related disease comprising measuring the level of at least one miRNA selected from Table 1 in a test biological sample from said subject, wherein the miRNA expression level is associated with an adverse prognosis; and an alteration in the level of the at least one miRNA in the test biological sample, relative to the level of a corresponding miRNA in a control biological sample, is indicative of an adverse prognosis.

Provided herein is a method of determining the prognosis of a subject with a brain tumor or related disease comprising measuring the level of at least one miRNA selected from Table 2 in a test biological sample from said subject, wherein the miRNA expression level is associated with an adverse prognosis; and an alteration in the level of the at least one miRNA in the test biological sample, relative to the level of a corresponding miRNA in a control biological sample, is indicative of an adverse prognosis.

In one embodiment, a biological sample can include, but is not limited to, a tissue sample containing cancer cells, biopsy fluid, blood or urine.

In one embodiment, the method further comprises obtaining or storing the biological sample prior to determining the set of expression profiles.

In another embodiment, obtaining the biological sample comprises isolating a cell fraction from a biopsy.

In yet another embodiment, obtaining the biological sample comprises isolating a cell fraction from a whole blood sample from the patient.

In yet another embodiment, a biological sample comprises an enriched cell fraction. For example, a cell fraction may be enriched for lymphocytes.

In one aspect, the plurality of brain tumor marker genes comprises at least three, at least five, or at least ten of the brain tumor marker genes listed in Table 1. In another aspect, at least 30%, at least 50%, at least 75%, or at least 90% of the genes are selected from the brain tumor marker genes listed in Table 1.

In one aspect, the plurality of brain tumor marker genes comprises at least three, at least five, or at least ten of the brain tumor marker genes listed in Table 2. In another aspect, at least 30%, at least 50%, at least 75%, or at least 90% of the genes are selected from the brain tumor marker genes listed in Table 2.

In another aspect, providing a diagnosis comprises providing a providing a probability score or a classification. In any of such methods, the diagnoses may indicate that the subject has a high risk of a brain tumor, further prescribing or providing a prophylactic therapy for reducing the risk of, or treating the brain tumor.

Prophylactic therapy encompasses administration of one or more anti-cancer therapies as described herein.

A brain tumor to be assessed utilizing the methods described herein can be, for example, glioblastoma multiforme.

Provided herein is a method of treating a brain tumor or related disease in a subject, comprising: (a) determining the amount of at least one miRNA selected from the genes in Table 1 in cancer cells, relative to control cells; and (b) altering the amount of miRNA expressed in the cells by (i) administering to the subject an effective amount of at least one isolated miRNA, a precursor thereof, an isolated variant thereof, or a biologically active fragment thereof, or (ii) administering to the subject an effective amount of at least one compound for inhibiting expression of the at least one miRNA. In such methods, determining the amount of at least one miRNA selected from the genes in Table 1 in cancer cells, relative to control cells comprises (i) comparing (a) a set of expression profiles of brain tumor marker genes in a biological sample from the subject, the set comprising expression profiles of a plurality of brain tumor marker genes from Table 1, to (b) a set of expression profiles of brain tumor marker genes in a biological sample from a control subject. In one embodiment, the method further comprises obtaining the set of expression profiles prior to the comparing step. In one embodiment, cancer cells are obtained from a tissue sample containing cancer cells, biopsy fluid, blood or urine. In one embodiment, at least 30%, at least 50%, at least 75%, or at least 90% of the genes are selected from the brain tumor marker genes listed in Table 1.

Provided herein is a method of treating a brain tumor or related disease in a subject, comprising: (a) determining the amount of at least one miRNA selected from the genes in Table 2 in cancer cells, relative to control cells; and (b) altering the amount of miRNA expressed in the cells by (i) administering to the subject an effective amount of at least one isolated miRNA, a precursor thereof, an isolated variant thereof, or a biologically active fragment thereof, or (ii) administering to the subject an effective amount of at least one compound for inhibiting expression of the at least one miRNA. In such methods, determining the amount of at least one miRNA selected from the genes in Table 2 in cancer cells, relative to control cells comprises (i) comparing (a) a set of expression profiles of brain tumor marker genes in a biological sample from the subject, the set comprising expression profiles of a plurality of brain tumor marker genes from Table 2, to (b) a set of expression profiles of brain tumor marker genes in a biological sample from a control subject. In one embodiment, the method further comprises obtaining the set of expression profiles prior to the comparing step. In one embodiment, cancer cells are obtained from a tissue sample containing cancer cells, biopsy fluid, blood or urine. In one embodiment, at least 30%, at least 50%, at least 75%, or at least 90% of the genes are selected from the brain tumor marker genes listed in Table 2.

In one embodiment, cancer cells comprise cells obtained from a tissue sample containing cancer cells, biopsy fluid, blood or urine.

In another embodiment, a brain tumor to be treated using the methods described herein is, for example, glioblastoma multiforme.

Methods of treatment may further comprise administering to a subject one or more therapeutic regimens.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an illustrative graph of hsa-mir-433 expression obtained from various GBM patient samples relative to control samples. Differential gene expression was observed: black shaded columns represent normal brain tissues (NGRL series) and hatched columns represent brain cancer samples (SN series).

FIG. 2 is an illustrative graph of quantitative PCR analysis of hsa-mir15b expression obtained from various GBM patient samples relative to control samples. Differential gene expression was observed: black shaded columns represent normal brain tissues (NGRL series) and hatched columns represent brain cancer samples (SN series).

FIG. 3 is an illustrative graph of quantitative PCR analysis of hsa-mir1 expression obtained from various GBM patient samples relative to control samples. Differential gene expression was observed: black shaded columns represent normal brain tissues (NGRL series) and hatched columns represent brain cancer samples (SN series).

FIG. 4 is an illustrative graph of quantitative PCR analysis of hsa-mir-127-3p from various GBM patient samples relative to control samples. Differential gene expression was observed: black shaded columns represent normal brain tissues (NGRL series) and hatched columns represent brain cancer samples (SN series).

FIG. 5 is an illustrative graph of quantitative PCR analysis of mmu-mir-124 from various GBM patient samples relative to control samples. Differential gene expression was observed: black shaded columns represent normal brain tissues (NGRL series) and hatched columns represent brain cancer samples (SN series).

FIG. 6 is an illustrative graph of quantitative PCR analysis of hsa-mir-15b from various GBM patient samples relative to control samples. Differential gene expression was observed: black shaded columns represent normal brain tissues (NGRL series) and hatched columns represent brain cancer samples (SN series).

### DETAILED DESCRIPTION OF THE INVENTION

While preferred embodiments of the present invention are shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention in one aspect relates generally to the identification, provision and use of a plurality of biomarkers to provide risk assessment of a subject for a brain tumor or a related disease, and products and processes related thereto. In one aspect, a plurality of biomarkers as described herein is provided to determine a risk for a brain tumor or a related disease. In one aspect are methods for determining a risk of a brain tumor or a related disease in a subject. In another aspect are methods of predicting the likelihood of brain cancer or a related disease in a subject. In yet another aspect are methods for identifying subjects at risk of brain cancer or a related disease, and kits for use in the method. In yet another aspect are nucleic acid arrays comprising nucleic acid probes that hybridize to brain tumor marker genes.

As used herein, a “biomarker” is an indicator of a particular disease state or state of a subject. As a non-limiting example, the biomarker is a gene.

Tumors are classified according to the kind of cell from which the tumor seems to originate. The most common primary brain tumor in adults comes from cells in the brain called astrocytes that make up the blood-brain barrier and contribute to the nutrition of the central nervous system. These tumors are called gliomas (astrocytoma, anaplastic astrocytoma, or glioblastoma multiforme) and account for 65% of all primary central nervous system tumors. Some of the tumors are, but not limited to, Oligodendroglioma, Ependymoma, Meningioma, Lymphoma, Schwannoma, and Medulloblastoma.

As used herein, the terms “tumor cells,” “transformed cells” or “cancer cells” refer to cells that have spontaneously converted to a state of unrestrained growth, i.e., they have acquired the ability to grow through an indefinite number of divisions in culture. Cancer cells may be characterized by such terms as neoplastic, anaplastic and/or hyperplastic, with respect to their loss of growth control. For purposes of this invention, the terms “transformed phenotype of malignant mammalian cells” and “transformed phenotype” are intended to encompass, but not be limited to, any of the following phenotypic traits associated with cellular transformation of mammalian cells: immortalization, morphological or growth transformation, and tumorigenicity, as detected by prolonged growth in cell culture, growth in semi-solid media, or tumorigenic growth in immuno-incompetent or syngeneic animals.

Brain and spinal cord tumors are abnormal growths of tissue found inside the skull or the bony spinal column, which are the primary components of the central nervous system (CNS). Benign tumors are non-cancerous, and malignant tumors are cancerous. The CNS is housed within rigid, bony quarters (i.e., the skull and spinal column), so any abnormal growth, whether benign or malignant, can place pressure on sensitive tissues and impair function. Tumors that originate in the brain or spinal cord are called primary tumors. Most primary tumors are caused by out-of-control growth among cells that surround and support neurons. In a small number of individuals, primary tumors may result from specific genetic disease (e.g., neurofibromatosis, tuberous sclerosis) or from exposure to radiation or cancer-causing chemicals. The cause of most primary tumors remains a mystery.

Brain cancer is a devastating disease and its most common form, glioblastoma multiforme, is responsible for 50% of all intracranial gliomas and 25% of intracranial tumors in adults. GBM diagnosis carries with it an average survival between twelve and eighteen months (with 90-95% patients surviving less than two years), without the possibility of spontaneous remission or effective treatment. The consistently short survival and absence of spontaneous remission that makes GBM such a devastating disease also render the evaluation of new therapies for this disease relatively rapid and unequivocal—overall survival represents the standard by which therapies for GBM are evaluated. Available treatment options include surgery, radiotherapy and chemotherapy.

### MicroRNA

MicroRNA genes are highly associated with chromosomal features involved in the etiology of different cancers. The perturbations in the genomic structure or chromosomal architecture of a cell caused by these cancer-associated chromosomal features can affect the expression of the miR gene(s) located in close proximity to that chromosomal feature. Evaluation of miR gene expression can therefore be used to indicate the presence of a cancer-causing chromosomal lesion in a subject. As the change in miR gene expression level caused by a cancer-associated chromosomal feature may also contribute to cancerigenesis, a given cancer can be treated by restoring the level of miR gene expression to normal. microRNA expression profiling can be used to diagnose cancer and predict whether a particular cancer is associated with an adverse prognosis. The identification of specific mutations associated with genomic regions that harbor miR genes in brain tumor patients provides a means for diagnosing brain tumors such as, for example, glioblastoma multiforme.

Small interfering RNA and miRNA have recently become of interest in biology and medicine due to their apparent roles in the regulation of gene expression via a process termed RNA interference (RNAi). The ability of organisms to dynamically respond to their environment is due in large part to regulation of gene expression. Regulation of gene expression is also important for the ability of multicellular organisms to generate the proper type and number of cells to create complex tissues and organs at the appropriate locations and times during development. Control of gene expression by a cell requires perception of environmental signals and appropriate response to these signals. Proteins have been studied extensively as mediators of these signals and a large number of protein-based regulators of gene expression are known. In contrast, the process of RNAi and, in particular, the role of miRNA in regulating gene expression is just beginning to be elucidated.

Micro-RNA molecules are produced as cleavage products of larger precursors that form self-complementary hairpin structures. The miRNA molecules are typically 21-23 nucleotides in length and are processed by a ribonuclease (such as Dicer in animals and DICER-LIKE1 in plants). A miRNA precursor can by polycistronic containing several different hairpin structures that each give rise to a different miRNA molecule. In contrast, small interfering RNA molecules are also generally about 21-23 nucleotides long but are produced from long hairpin precursors processed such that several different siRNA molecules can arise from a single hairpin structure.

Typically, miRNA hybridizes to a specific target mRNA through near complementary base pairing to form large complexes. Complex formation results in arrest of translation and/or increased degradation of the target mRNA.

As used herein, the term “small RNA” is intended to mean a ribonucleic acid having a length between about 20 and 30 nucleotides, and terminating in a 5′ phosphate and a 3′ hydroxyl. A 5′ phosphate is understood to be a (PO₄)₂— (PO₄H)— or (PO₄H₂) moiety covalently attached to the 5′ carbon of ribose via one of the oxygens. A 3′ hydroxyl is understood to be an OH or O-moiety covalently attached to the 3′ carbon of ribose via the oxygen. Those skilled in the art will recognize that the presence or absence of hydrogens in the phosphate and hydroxyl moieties as listed above is a function of their pKa values and the pH of their environment. Most small RNA molecules are 20 to 25 nucleotides in length with a large majority being about 21 or 22 nucleotides long. However, small RNA molecules having longer sequences are also known including for example, those having a length of 26 nucleotides (see, for example, Hamilton et al., EMBO J. 21:4671 (2002)) or 28 nucleotides (see, for example, Mochizuki et al., Cell 110:689-99 (2002)).

Small RNA can be identified according to its function in a cell including, for example, having a non-coding sequence (i.e. not being translated into protein) and being capable of inhibiting expression of at least one mRNA. Small RNA can also be identified according to its biosynthesis. For example, a first type of small RNA, short interfering RNA (siRNA), is typically synthesized from endogenous or exogenous double stranded RNA (dsRNA) molecules having hairpin structures and processed such that numerous siRNA molecules are produced from both strands of the hairpin. In contrast, micro-RNA molecules are typically produced from endogenous dsRNA molecules having one or more hairpin structure such that a single micro-RNA molecule is produced from each hairpin structure. The terms “small RNA,” “siRNA” and “micro-RNA” are intended to be consistent with their use in the art as described, for example, in Ambros et al., RNA 9: 277-279 (2003).

A small RNA can be distinguished from mRNA based on the presence of a 5′ cap structure in mRNA and absence of the cap structure in small RNA. The 5′ cap structure typically found in eukaryotic mRNA is a 7-methylguanylate having a 5′ to 5′ triphosphate linkage to the terminal nucleotide. Small RNA can also be distinguished from mRNA based on the presence of a terminal polyadenylate sequence at the 3′ end of mRNA which is absent in small RNA.

As used herein, a “miR gene product” or “miRNA” also means the unprocessed or processed RNA transcript from a miR gene. As the miR gene products are not translated into a protein, the term “miR gene products” does not include proteins.

### Expression Profiles of GBM

Without limiting the scope of the present invention, any number of techniques known in the art can be employed for expression profiling of GBM biomarkers.

In some embodiments, the detecting step(s) comprises use of a detection assay including, but not limited to, sequencing assays, polymerase chain reaction assays, hybridization assays, hybridization assay employing a probe complementary to a mutation, fluorescent in situ hybridization (FISH), nucleic acid array assays, bead array assays, primer extension assays, enzyme mismatch cleavage assays, branched hybridization assays, NASBA assays, molecular beacon assays, cycling probe assays, ligase chain reaction assays, invasive cleavage structure assays, ARMS assays, and sandwich hybridization assays. In some preferred embodiments, the detecting step is carried out using cell lysates. In some embodiments, the methods may comprise detecting a second nucleic acid target. In some preferred embodiments, the second nucleic acid target is RNA. In some particularly preferred embodiments, the second nucleic acid target may be, for example, U6 RNA or GAPDH mRNA.

Provided herein are methods that can be employed for expression profiling of GBM biomarkers. One non-limiting measure of change in gene expression is to assess fold increase or decrease of a gene compared to background levels. For example, in one embodiment, one can detect genes that exhibit an about 2-fold increase above or an about 2-fold decrease below, background. In another embodiment, one can detect genes that exhibited a fold increase or decrease above background of at least about 3-fold, and in another embodiment at least about 4-fold, and in another embodiment at least about 5-fold, and in another embodiment at least about 6-fold, and in another embodiment at least about 7-fold, and in another embodiment at least about 8-fold, and in another embodiment at least about 9-fold, and in another embodiment at least about 10-fold or higher changes. Fold increases or decreases are not typically compared from one gene to another, but rather, with reference to background level for that particular gene.

In one aspect of the method of the present invention, the expression profile can include the expression of one or more of the genes disclosed herein. Expression of transcripts is measured, for example, using the methods described herein.

For RNA expression, methods include but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene.

In addition to general expression of a gene, the number of copies of a gene in a cell can be determined with nucleic acid probes to the genes. In one embodiment, fluorescent in situ hybridization (FISH) can be used to detect the number of copies of a gene in a cell. Established hybridization techniques such as FISH are contemplated herein. In one embodiment, the number of genes within a peripheral blood cell is determined using a FISH assay for a plurality of GBM markers disclosed herein.

Nucleic acid arrays are particularly useful for detecting the expression of the genes of the present invention. The production and application of high-density arrays in gene expression monitoring have been disclosed previously in, for example, WO 97/10365; WO 92/10588; U.S. Pat. No. 6,040,138; U.S. Pat. No. 5,445,934; or WO95/35505, all of which are incorporated herein by reference in their entireties. Also for examples of arrays, see Hacia et al. (1996) Nature Genetics 14:441-447; Lockhart et al. (1996) Nature Biotechnol. 14:1675-1680; and De Risi et al. (1996) Nature Genetics 14:457-460. In general, in an array, an oligonucleotide, a cDNA, or genomic DNA, that is a portion of a known gene, occupies a known location on a substrate. A nucleic acid target sample is hybridized with an array of such oligonucleotides and then the amount of target nucleic acids hybridized to each probe in the array is quantified. One preferred quantifying method is to use confocal microscope and fluorescent labels. The Affymetrix GeneChip™ Array system (Affymetrix, Santa Clara, Calif.) and the Atlas™ Human cDNA Expression Array system are particularly suitable for quantifying the hybridization. It will be apparent, however, that any similar systems or other effectively equivalent detection methods can also be used. In a particularly preferred embodiment, one can use the genes described herein to design novel arrays of polynucleotides, cDNAs or genomic DNAs for screening methods described herein. Such novel pluralities of polynucleotides are contemplated to be a part of the present invention and are described in detail below.

Suitable nucleic acid samples for screening on an array contain transcripts of interest or nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like. In one embodiment, nucleic acids for screening are obtained from a homogenate of cells or tissues or other biological samples. In another embodiment, a sample is a total RNA preparation of a biological sample. In yet another embodiment, such a nucleic acid sample is the total mRNA isolated from a biological sample.

In one embodiment, a nucleic acid sample may be amplified prior to hybridization. One of skill in the art will appreciate that whatever amplification method is used, if a quantitative result is desired, a method that maintains or controls for the relative frequencies of the amplified nucleic acids to achieve quantitative amplification may be used. Methods of quantitative amplification include, but are not limited to, quantitative PCR involves simultaneously co-amplifying a known quantity of a control sequence using the same primers. This provides an internal standard that may be used to calibrate the PCR reaction. The high-density array may then include probes specific to the internal standard for quantification of the amplified nucleic acid. Other suitable amplification methods include, but are not limited to, polymerase chain reaction (PCR) (see Innis, et al., PCR Protocols, A Guide to Methods and Application, Academic Press, Inc. San Diego, (1990)); ligase chain reaction (LCR) (see Wu and Wallace, Genomics, 4: 560 (1989), Landegren, et al., Science, 241:1077 (1988) and Barringer, et al., Gene, 89:117 (1990)); transcription amplification (Kwoh, et al., Proc. Natl. Acad. Sci. USA, 86:1173 (1989)), and self-sustained sequence replication (Guatelli, et al., Proc. Nat. Acad. Sci. USA, 87:1874 (1990)).

Nucleic acid hybridization simply involves contacting a probe and target nucleic acid under conditions where the probe and its complementary target can form stable hybrid duplexes through complementary base pairing. As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Laboratory Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., all of which are incorporated by reference herein in their entirety. Nucleic acids that do not form hybrid duplexes are washed away from the hybridized nucleic acids and the hybridized nucleic acids can then be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

High stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). One can use the formulae in Meinkoth et al., 1984, Anal. Biochem. 138, 267-284 (incorporated herein by reference in its entirety) to calculate the appropriate hybridization and wash conditions to achieve desired levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In some embodiments, stringent hybridization conditions for DNA:DNA hybrids include hybridization at an ionic strength of 6×SSC (0.9 M Na⁺) at a temperature of between about 20° C. and about 35° C., more preferably, between about 28° C. and about 40° C., and even more preferably, between about 35° C. and about 45° C. In other embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C. These values are based on calculations of a melting temperature for molecules larger than about 100 nucleotides, 0% formamide and a G+C content of about 40%. Alternatively, Tm can be calculated empirically as set forth in Sambrook et al., supra, pages 9.31 to 9.62.

The hybridized nucleic acids are detected by detecting one or more labels attached to the sample nucleic acids. The labels may be incorporated by any of a number of means well known to those of skill in the art. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horseradish peroxidase, alkaline phosphatase and others commonly used in an ELISA), and colorimetric labels such as colloidal gold or colored glass or plastic (e.g., polystyrene, polypropylene, latex, etc.) beads. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

In some embodiments of the present invention, detection structures are detected using a hybridization assay. In a hybridization assay, the presence of absence of a given nucleic acid sequence is determined based on the ability of the DNA from the sample to hybridize to a complementary DNA molecule (e.g., an oligonucleotide probe). A variety of hybridization assays using a variety of technologies for hybridization and detection are available and include, but are not limited, to those described herein.

The complement of a nucleic acid sequence as used herein refers to an oligonucleotide which, when aligned with the nucleic acid sequence such that the 5′ end of one sequence is paired with the 3′ end of the other, is in “anti-parallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

In some embodiments, hybridization of a probe to the sequence of interest (e.g., a SNP or mutation) is detected directly by visualizing a bound probe (e.g., a Northern or Southern assay; see e.g., Ausabel et al. (eds.), Current Protocols in Molecular Biology, John Wiley & Sons, NY [1991]). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition of low, medium, or high stringency conditions. Unbound probe is removed and the presence of binding is detected by visualizing the labeled probe.

For analysis by Northern blotting, total RNA isolation is performed by acid guanidinium thiocyanate-phenol-chloroform extraction. Northern analysis is performed as described according to standard protocols, except that the total RNA is resolved on a 15% denaturing polyacrylamide gel, transferred onto Hybond-N+membrane (Amersham Pharmacia Biotech), and the hybridization and wash steps are performed at 50° C. Oligodeoxynucleotides used as Northern probes are 5′-32P-phosphorylated, complementary to the miRNA sequence and 20 to 25 nt in length. 5S rRNA is detected by ethidium staining of polyacrylamide gels prior to transfer. Blots are stripped by boiling in 0.1% aqueous sodium dodecylsulfate/0.1×SSC (15 mM sodium chloride, 1.5 mM sodium citrate, pH 7.0) for 10 min, and are re-probed up to 4 times until the 21-nt signals become too weak for detection. Finally, blots are probed for val-tRNA as size marker.

In some embodiments of the present invention, variant sequences are detected using a DNA chip hybridization assay. In this assay, a series of oligonucleotide probes are affixed to a solid support. The oligonucleotide probes are designed to be unique to a given target sequence (e.g., miRNA target sequence). The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) may be utilized (see e.g., U.S. Pat. Nos. 6,017,696; 6,068,818; and 6,051,380; each of which is herein incorporated by reference). Through the use of microelectronics, Nanogen's technology enables the active movement and concentration of charged molecules to and from designated test sites on its semiconductor microchip. DNA capture probes unique to a given target sequence are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target sequences by determining which of the DNA capture probes hybridize, with target sequences. An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on an x/Y translation stage under a set of four piezoelectric nozzles, one for each of the four standard DNA bases. The translation stage moves along each of the rows of the array and the appropriate reagent is delivered to each of the reaction sites. For example, the A amidite is delivered only to the sites where amidite A is to be coupled during that synthesis step and so on. Common reagents and washes are delivered by flooding the entire surface and then removing them by spinning.

DNA probes unique for the target sequence (e.g., miRNA target sequence) of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a bead array technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the bead array is contacted with a prepared subject sample (e.g., nucleic acid sample). Hybridization is detected using any suitable method.

In some embodiments of the present invention, hybridization is detected by enzymatic cleavage of specific structures.

In some embodiments, hybridization of a bound probe is detected using a TaqMan® assay (PE Biosystems, Foster City, Calif.; See e.g., U.S. Pat. Nos. 5,962,233 and 5,538,848, each of which is herein incorporated by reference). The assay is performed during a PCR reaction. The TaqMan® assay exploits the 5′-3′ exonuclease activity of the AMPLITAQ GOLD® DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD® polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the target sequence location. Incorporation of the label into the DNA can be detected by any suitable method (e.g., if the nucleotide contains a biotin label, detection is via a fluorescently labeled antibody specific for biotin).

Additional detection assays useful in the detection of miRNA detection structures include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay, PNAS USA 88, 189-93 (1991)); and sandwich hybridization methods (e.g., U.S. Pat. No. 5,288,609, herein incorporated by reference in its entirety).

The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

### Multimarker Classifier

Multimarker classifiers can be utilized to classify a brain tumor or a related disease. In one embodiment, the multimarker classifier is obtained by a comparison of expression levels of genes in a plurality of subjects having a brain tumor or a related disease to expression levels of genes in a plurality of subjects who do not have a brain tumor or a related disease, and identifying genes that are statistically significantly differentially expressed between the two pluralities.

In one aspect of the invention, the multimarker classifier comprises a plurality or all of the GBM genes identified in Table 1, which genes have been identified as differentially expressed miRNAs between GBM brain compared to normal brain.

TABLE 1
miRNA name

| | SEQ ID |
|---|---|
| miRNA | NO |
| hsa-miR-15b | 1 |
| hsa-miR-23a | 2 |
| hsa-miR-146b | 3 |
| hsa-miR-23b | 4 |
| hsa-miR-130a | 5 |
| hsa-miR-199a | 6 |
| hsa-miR-191 | 7 |
| hsa-miR-25 | 8 |
| hsa-miR-1 | 9 |
| hsa-miR-9 | 10 |
| hsa-miR-10a | 11 |
| hsa-miR-629 | 12 |
| hsa-miR-1262 | 13 |
| hsa-miR-452 | 14 |
| hsa-miR-1827 | 15 |
| hsa-miR-628-5p | 16 |
| hsa-miR-181a-2 | 17 |
| miR-127-3p | 18 |
| hsa-miR-433 | 19 |
| hsa-miR-219 | 20 |
| hsa-miR-886-5p | 21 |
| hsa-miR-21 | 22 |
| hsa-miR-21 | 23 |
| hsa-miR-142-5p | 24 |
| hsa-miR-92b | 25 |
| hsa-miR-15a | 26 |
| hsa-miR-106b | 27 |
| hsa-miR-27a | 28 |
| hsa-miR-365 | 29 |
| hsa-miR-143 | 30 |
| hsa-miR-186 | 31 |
| hsa-miR-34a | 32 |
| hsa-miR-199b-3p | 33 |
| hsa-miR-1274b | 34 |
| hsa-miR-34c-5p | 35 |
| hsa-miR-223 | 36 |
| hsa-miR-204 | 37 |
| hsa-miR-99b | 38 |
| hsa-miR-31 | 39 |
| hsa-miR-532-5p | 40 |
| hsa-miR-1308 | 41 |
| hsa-miR-210 | 42 |
| hsa-miR-16 | 43 |
| hsa-miR-126 | 44 |
| hsa-miR-24 | 45 |
| hsa-miR-598 | 46 |
| hsa-miR-374b | 47 |
| hsa-miR-184 | 48 |
| hsa-miR-330-3p | 49 |
| hsa-miR-9 | 50 |
| hsa-miR-760 | 51 |
| hsa-miR-379 | 52 |
| hsa-miR-7 | 53 |
| hsa-miR-128 | 54 |
| hsa-miR-543 | 55 |
| hsa-miR-485-5p | 56 |
| hsa-miR-499-5p | 57 |
| hsa-miR-129-3p | 58 |
| hsa-miR-124 | 59 |
| hsa-miR-219-2-3p | 60 |

The genes in Table 1 represent genes which have the potential to discriminate between subjects having brain cancer and those who do not. In certain embodiments, a plurality of genes selected from those identified in Table 1 are used with the products and methods described and claimed herein to discriminate between (identify) subjects having brain cancer and those who do not. In some embodiments of the invention, a plurality of genes selected from the genes identified in Table 1 are used with the products and methods described and claimed herein to determine a risk of, or predict the likelihood of, GBM. In yet other embodiments of the invention, a plurality of genes selected from the genes identified in Table 1 are used with the products and methods described and claimed herein to determine the severity of GBM.

In yet other embodiments of the invention, a plurality of genes selected from the genes identified in Table 1 are used with the products and methods described and claimed herein to determine the severity of GBM.

In another aspect of the invention, the multimarker classifier comprises a plurality or all of the GBM genes identified in Table 2.

TABLE 2
miRNA name

| | |
|---|---|
| Hsa-mir-433 | Hsa-mir-1 |
| Hsa-mir-15b | Hsa-mir-127 |
| Mmu-mir-124 | |

The genes in Table 2 represent a subset of genes of Table 1 which have the potential to discriminate between subjects having brain cancer and those who do not. In certain embodiments, a plurality of genes selected from those identified in Table 2 are used with the products and methods described and claimed herein to discriminate between (identify) subjects having brain cancer and those who do not. In some embodiments of the invention, a plurality of genes selected from the genes identified in Table 2 are used with the products and methods described and claimed herein to determine a risk of, or predict the likelihood of, for example, GBM. In yet other embodiments of the invention, a plurality of genes selected from the genes identified in Table 2 are used with the products and methods described and claimed herein to determine the severity of GBM.

In yet other embodiments of the invention, a plurality of genes selected from the genes identified in Table 2 are used with the products and methods described and claimed herein to determine the severity of GBM.

As used herein, a biological sample to be assessed refers to, but is not limited to, solid tissue (e.g. tumor biopsy material: such as fixed, paraffin-embedded, or fresh, or frozen tissues, which can be obtained from fine needle, core, or other types of biopsy, such as, for example, by fine needle aspiration), viscous liquids (e.g. lymph gland contents) and biological fluids (e.g. blood, urine, cerebrospinal fluid and ascites fluid). Cellular constituents, including RNA and protein, derived from tumor cells have been found in biological fluids of cancer patients. Circulating nucleic acids and proteins may result from tumor cell lysis and can be subjected to expression analysis.

### Glioblastoma Multiforme Prognosis

Glioblastoma multiforme prognosis can be divided into three subgroups dependent on Karnofsky Performance Score (KPS), the age of the patient, and treatment:

| | | Historical | | | |
|---|---|---|---|---|---|
| | | Median | Historical | Historical | Historical |
| | | Survival | 1-Year | 3-Year | 5-Year |
| RPA class | Definition | Time | Survival | Survival | Survival |
| III | Age <50, KPS >=90 | 17.1 months | 70% | 20% | 14% |
| IV | Age <50, KPS <90 | 11.2 months | 46% | 7% | 4% |
| | Age >50, KPS >=70, | | | | |
| | surgical removal with | | | | |
| | good neurologic | | | | |
| | function | | | | |
| V + VI | Age >=50, KPS >=70, | 7.5 months | 28% | 1% | 0% |
| | surgical removal | | | | |
| | with poor neurologic | | | | |
| | function | | | | |
| | Age >=50, KPS >=70, | | | | |
| | no surgical | | | | |
| | removal | | | | |
| | Age >=50, KPS <70 | | | | |

The Karnofsky score runs from 100 to 0, where 100 is “perfect” health and 0 is death. Although the score has been described with intervals of 10, a practitioner may choose decimals if he or she feels a patient's situation holds somewhere between two marks: 100%—normal, no complaints, no signs of disease; 90%—capable of normal activity, few symptoms or signs of disease; 80%—normal activity with some difficulty, some symptoms or signs; 70%—caring for self, not capable of normal activity or work; 60%—requiring some help, can take care of most personal requirements; 50%—requires help often, requires frequent medical care; 40%—disabled, requires special care and help; 30%—severely disabled, hospital admission indicated but no risk of death; 20%—very ill, urgently requiring admission, requires supportive measures or treatment; 10%—moribund, rapidly progressive fatal disease processes; 0%—death.

Provided herein is a method of determining the prognosis of a subject with a brain tumor comprising measuring the level of at least one miRNA selected from Table 1 in a test sample from said subject, wherein the miRNA expression level is associated with an adverse prognosis; and an alteration in the level of the at least one miRNA in the test sample, relative to the level of a corresponding miRNA in a control sample, is indicative of an adverse prognosis. In one aspect, the test sample is a brain sample.

Provided herein is a method of determining the prognosis of a subject with a brain tumor comprising measuring the level of at least one miRNA selected from Table 2 in a test sample from said subject, wherein the miRNA expression level is associated with an adverse prognosis; and an alteration in the level of the at least one miRNA in the test sample, relative to the level of a corresponding miRNA in a control sample, is indicative of an adverse prognosis. In one aspect, the test sample is a brain sample.

A brain tumor to be assessed using methods described herein can be, for example, glioblastoma multiforme. In one aspect, the at least one miRNA detected in a test sample can be expressed in increased levels compared to a control sample. Alternatively, the at least one miRNA detected in a test sample can be expressed in decreased levels compared to a control sample.

Provided herein is a method for identifying a subject having a brain tumor, comprising determining expression profiles of no more than five to five hundred genes in a biological sample containing cells from a subject, wherein at least 20% of the genes are selected from the brain tumor marker genes listed in Table 1.

Provided herein is a method for identifying a subject having a brain tumor, comprising determining expression profiles of no more than five to five hundred genes in a biological sample containing cells from a subject, wherein at least 20% of the genes are selected from the brain tumor marker genes listed in Table 2.

Also provided herein is a method of determining the prognosis of a subject with a brain tumor comprising measuring the level of at least one miRNA selected from Table 1 in a test biological sample from said subject, wherein the miRNA expression level is associated with an adverse prognosis; and an alteration in the level of the at least one miRNA in the test biological sample, relative to the level of a corresponding miRNA in a control biological sample, is indicative of an adverse prognosis.

Also provided herein is a method of determining the prognosis of a subject with a brain tumor comprising measuring the level of at least one miRNA selected from Table 2 in a test biological sample from said subject, wherein the miRNA expression level is associated with an adverse prognosis; and an alteration in the level of the at least one miRNA in the test biological sample, relative to the level of a corresponding miRNA in a control biological sample, is indicative of an adverse prognosis.

Such methods may further comprise obtaining or storing the biological sample prior to determining the set of expression profiles.

A biological sample may be, for example, a tissue sample containing cancer cells, biopsy fluid, blood or urine. Obtaining the biological sample may include isolating a cell fraction from a biopsy. Alternatively, obtaining the biological sample may include isolating a cell fraction from a whole blood sample from the patient. In some embodiments, a biological sample comprises an enriched cell fraction. In other embodiments, a cell fraction is enriched for lymphocytes.

Providing a diagnosis may include providing a providing a probability score and/or a classification.

In any of such methods, the plurality of brain tumor marker genes comprises at least three, at least five, or at least ten of the brain tumor marker genes listed in Table 1.

In any of such methods, the plurality of brain tumor marker genes comprises at least three, at least five, or at least ten of the brain tumor marker genes listed in Table 2.

In one embodiment, a diagnosis indicates that the subject has a high risk of a brain tumor, further prescribing or providing a prophylactic therapy for reducing the risk of, or treating the brain tumor. Prophylactic therapy includes administration of one or more anti-cancer therapies.

Furthermore, in any of such methods, at least 30%, at least 50%, at least 75%, or at least 90%, of the genes are selected from the brain tumor marker genes listed in Table 1.

Furthermore, in any of such methods, at least 30%, at least 50%, at least 75%, or at least 90%, of the genes are selected from the brain tumor marker genes listed in Table 2.

A brain tumor to be diagnosed and treated is, for example, glioblastoma multiforme.

### Multimarker Classifier

The expression levels of a plurality of genes in the multimarker classifier from a plurality of subjects who do not have GBM, and the expression levels of a plurality of genes in the multimarker classifier from a plurality of subjects who do have GBM, may be determined In one example, a representative data set of samples from a plurality of subjects who do not have GBM and from a plurality of subjects who do not have GBM is collected. In another example, samples from subjects meeting the definition and phenotypic sub-classification of GBM based on criteria advocated by Karnofsky Performance Scoring (KPS) can be taken.

In one embodiment, to minimize potential confounding by race/ethnicity and age, and to optimize statistical power of the classifier, analyses can be restricted to particular races or ethnicities and ages. Identical exclusion/selection and frequency matching criteria can be used to select participants for independent validation analyses.

Specimens for analysis for the multi-marker classifier can be selected using, for example, a nested case-control study design. For example, all GBM cases in the study population are identified. A balanced random sample of moderate cases to achieve approximately equal proportions of GBM and non-GBM cases are also identified. Controls are frequency matched on race (e.g., within same race) and age at sample collection (e.g., within 5 years).

The expression profile of the genes for GBM genes can be determined by any of the methods known in the art and described above.

In one embodiment, analysis of the expression profiles that make up the multimarker classifier can be conducted using natural log-transformed data. For example, both supervised and unsupervised approaches may be used to identify inherent differences in gene expression patterns between GBM cases and controls. Unsupervised methods, such as cluster or principal component analysis (PCA), or any other methods in microarray analyses, may be used. PCA may be used to reduce the high dimension microarray data to 2 or 3 dimensions for easy visualization thus allowing similar comparisons across samples. In one embodiment, cluster analyses may simultaneously group samples and genes that share similar expression patterns. The color representation of heat mapping from cluster analysis can be used to reveal unique gene signatures to distinguish various sub-groups of participants in a global genomic fashion. A phylogenetic tree of genes that are differentially expressed may be constructed, e.g., by Cluster or TreeView software, or a hierarchical clustering algorithm that utilizes the Pearson's correlation coefficient, for example.

In another embodiment, supervised approaches can be used to identify subsets of genes that can robustly distinguish GBM cases from controls. Support vector machine (SVM), the significance analysis of microarrays (SAM), and the Shrunken Centroids methods, and other available methods can be used to classify disease status. Briefly, in SAM analysis, a score statistic is calculated for each gene based on a ratio of change in gene expression (numerator) to standard deviation in the data for that gene plus an adjustment to minimize the coefficient of variation and enable comparison across all genes (denominator). In another embodiment, permutations to estimate the percentage of genes identified by chance, false discovery rate (FDR), for genes with scores greater than an adjustable threshold are also used. The FDR, q-value of a selected gene corresponds to the FDR for the gene list that includes the gene and all genes that are more significant. In another embodiment, a direct approach to gene selection to build classifiers using a subset of genes in a SVM model may be used. For example, the RankGene system can be used to choose K genes with the largest absolute value of scores in an SVM model. The system takes into account several criteria such as t-test statistic, information gain, and variance of expression to determine the discriminative strength of individual genes.

Other analytical approaches to gene selection can also be used: for example, those that reduce the possibility of colinearity among the selected K genes to increase classifier performance. Other non-limiting examples, such as, for example, greedy forward selection, genetic algorithms, and/or gradient-based leave-one-out gene selection (GLGS) algorithms can be used.

In one embodiment, one criterion for classifier gene selection may be defined a priori. For example, in certain embodiments, genes that satisfy the following three criteria in comparisons between GBM cases and controls can comprise the set of genes used in a particular embodiment: (1) Student's t-test p-value<0.001; (2) fold change differences≧2.0; and (3), false discovery rates (FDR)≦10% as using (SAM). Standards advocated by the Karnofsky Performance Score (KPS) may also be followed.

In another embodiment, the performance of the classifier may be evaluated. For example, cross validation approaches such as the 10-fold cross validation approach may be used. In this approach, derivation data is divided into 10 equal parts, each with 12 samples. 11 parts of the data are selected as a “test or training set” from which a classification model with K gene can be constructed to confirm its prediction performance on the remaining excluded part. The decision call for each excluded sample tested can be made based on the prediction function/score provided by each method. For instance, the Shrunken Centroids methods can provide a predictive probability of being in the GBM group. The procedure can be repeated 12 times, and then the overall error rate can be estimated. The overall error will likely depend on the number of K genes in the model. Hence, this number may be varied by changing the tuning parameter when using the Shrunken Centroids method. The optimal number of genes, K, or equivalently the optimal tuning parameter may be chosen such that the overall error rate reaches its minimum. Permutation testing may be used to assess the significance of the observed error rate. Briefly, 60 samples will be randomly relabeled as belonging to the GBM group and the remaining 60 in the term control group. The same 10-fold cross validation analysis as previously described may be conducted, and overall error rates recorded based on the optimal K genes from this permuted data. This procedure may be repeated as necessary, e.g., 10, 100, 1,000, 5,000 times (or any number in between) to obtain a null distribution of the overall error rate. Any other methods to measure the significance of overall error rates in the derivation set with correct classification may be used. For example, methods that can trade off bias for low variance, such as balance bootstrap re-sampling approaches, which have been shown to be a variance reducing technique, may also be used.

In another embodiment of the present invention, microarray findings are confirmed, e.g., using quantitative RT-PCR (qRT-PCR) methods. As a non-limiting example, a plurality of genes (e.g., 1, 2, 3, 4, 5, up to 50, or any number in between; preferably, 1-20 genes) may be selected for confirmation using methods such as qRT-PCR. qRT-PCR for the selected genes in the derivation set can be performed on all samples in both the derivation and the validation set. Correlation coefficients (e.g., Spearman's correlation coefficients) of expression values from microarray and qRT-PCR approaches can then be assessed.

In another embodiment of the present invention, the observed error rate for the samples in the validation data set can be calculated based on the classifier constructed from the independent samples from the derivation data set. A GBM status label may be permuted on the derivation set to obtain a null classifier and validate its prediction performance on the validation data set. This procedure may be repeated as necessary, e.g., 10, 100, 1,000, 5,000 times (or any number in between) to obtain significance levels of the observed error rates. Alternatively, other methods of testing classification accuracy, such as PCA and multi-dimensional scaling (MDS) may be used. In one embodiment, a 2-(GBM versus non-GBM) or 3-dimensional PCA of the validation samples based on the K genes in the classifier may be constructed from the derivation set.

In another embodiment, bioinformatics approaches may be used to retrieve and interpret complex biological interactions of the multimarker classifier. For example, Database for Annotation, Visualization and Integrated Discovery (DAVID) and Ingenuity Pathway Analysis (IPA) software (Ingenuity, Redwood City, Calif.) may be used to study systems biology and to explore mechanistic hypotheses. For example, an analysis based on DAVID can provide a comprehensive set of functional annotation tools and an enrichment analytic algorithm technique to identify enriched functional-related gene groups. A modified Fisher Exact p-value, an EASE score, can be used to measure the gene-enrichment in annotation terms by comparing the proportion of genes that fall under each category or term to the human genome background. An overall enrichment score for the group can be derived as the geometric mean (in log scale) of members' p-values (EASE score) in a corresponding annotation cluster. As another example, using analysis based on IPA, Ingenuity Pathways Knowledge Base (IPKB), a published and peer-reviewed database and computational algorithms can be used to identify local networks that are particularly enriched for the Network Eligible Genes, which can be defined as genes in our list of differentially expressed genes with at least one previously defined connection to another gene in the IPKB. A score that takes into account the number of Network Eligible Genes and the size of the networks, can be calculated using a Fisher Exact test as the negative log of the probability that the genes within that network are associated by chance. For example, a score of 3 (p-value corresponding to 0.001) as the cutoff for significance of the network can be used. The overall enrichment score in the analysis conducted using DAVID and the network score obtained in IPA can then be used to rank the biological significance of gene function clusters and networks, respectively, in PTD.

### Comparison

In one embodiment of the present invention, a set of expression profiles of GBM marker genes in a biological sample from a subject are compared to a multimarker classifier. As one example, the expression profile is determined prior to the comparing step. As one example, the expression profile is of at least 1, 2, 3, 4, 5, 6, 7, 8, 9, 10, 11, 12, 13, 14, 15, 16, 17, 18, 19, 20, 25, 30, 35, 40, 45, 50, 55, or 60 (or any number therein) of the GBM marker genes listed in Table 1. As another example, the expression profile is of at least 1, 2, 3, 4 or 5 of the GBM marker genes listed in Table 2.

In one example, the expression profile is of at least 1%, 5%, 10%, 20%, 30%, 40%, 50%, or between 1% and 50% of the GBM marker genes listed in Table 1. In another example, the expression profile is of 1 to 60, 10 to 50, 20 to 40, 1 to 15, 1 to 40, 1 to 30, 1 to 20 genes, 2 to 19 genes, 3 to 18 genes, 4 to 17 genes, 5 to 16 genes, 6 to 17 genes, 8 to 15 genes, 9 to 14 genes, 10 to 13 genes, 11 to 12 genes, 5 to 20 genes, 5 to 10 genes, or any other number in between 1 to 60 genes of Table 1 in a biological sample. In another example, the expression profile is of at least 1%, 5%, 10%, 20%, 30%, 40%, 50%, or between 1% and 50% of the GBM marker genes listed in Table 2. In another example, the expression profile is of 1 to 5, 1 to 4, 2 to 4, or 3, or any other number in between 1 to 5 genes of Table 2 in a biological sample.

In the comparison of each gene in the expression profile to the same gene in the classifier, a gene identified as being upregulated or downregulated in a biological sample according to the invention may be regulated in the same direction and to at least about 5%, and more preferably at least about 10%, and more preferably at least 20%, and more preferably at least 25%, and more preferably at least 30%, and more preferably at least 35%, and more preferably at least 40%, and more preferably at least 45%, and more preferably at least 50%, and preferably at least 55%, and more preferably at least 60%, and more preferably at least 65%, and more preferably at least 70%, and more preferably at least 75%, and more preferably at least 80%, and more preferably at least 85%, and more preferably at least 90%, and more preferably at least 95%, and more preferably of 100%, or any percentage change between 5% and higher in 1% increments (i.e., 5%, 6%, 7%, 8% . . . ), of the level of expression of the gene that is seen in the multimarker classifier. A gene identified as being upregulated or downregulated in an expression profile according to the invention can also be regulated in the same direction and to a higher level than the level of expression of the gene that is seen in the multimarker classifier.

The values obtained from the biological sample and multimarker classifier are statistically processed using any suitable method of statistical analysis to establish a suitable baseline level using methods standard in the art for establishing such values. Statistical significance according to the present invention should be at least p<0.05.

One can appreciate that differences between expression of genes may be small or large. Some small differences may be very reproducible and therefore nonetheless useful. For other purposes, large differences may be desirable for ease of detection of the activity. It will be therefore appreciated that the exact boundary between what is called a positive result and a negative result can shift, depending on the goal of the screening assay and the genes to be screened. For some assays it may be useful to set threshold levels of change. One can readily determine the criteria for screening given the information provided herein.

The level of expression of the gene or genes detected in the biological sample of the invention is compared to the baseline or control level of expression of that gene in the multimarker classifier. More specifically, according to the present invention, a “baseline level” is a control level of biomarker expression in the multimarker classifier against which a test level of biomarker expression (i.e., in the biological sample) can be compared. In one embodiment, control expression levels of genes of the multimarker classifier have been predetermined, such as for the genes listed in Table 1. In another embodiment, control expression levels of genes of the multimarker classifier have been predetermined, such as for the genes listed in Table 2. Such a form of stored information can include, for example, but is not limited to, a reference chart, listing or electronic file of gene expression levels and profiles for GBM marker genes, or any other source of data regarding baseline biomarker expression that is useful in the methods disclosed herein. Therefore, it can be determined, based on the control or baseline level of biomarker expression or biological activity, whether the expression level of a gene or genes in a biological sample is/are more statistically significantly similar to the baseline multimarker classifier of GBM marker genes. A profile of individual gene markers, including a matrix of two or more markers, can be generated by one or more of the methods described herein. According to the present invention, a profile of the genes in a biological sample refers to a reporting of the expression level of a given gene from Table 1. Additionally or alternatively, according to the present invention, a profile of the genes in a biological sample refers to a reporting of the expression level of a given gene from Table 2. The data can be reported as raw data, and/or statistically analyzed by any of a variety of methods, and/or combined with any other prognostic marker(s).

### Providing a Risk Assessment

In one embodiment of the present invention, a risk assessment for GBM is provided. The risk assessment may be an output from the comparison of a set of expression profiles of GBM marker genes in a biological sample to a multimarker classifier, as described above. The risk assessment may provide a dichotomous output (yes/no), a probability score, or a risk classification, as non-limiting examples. For example, the risk assessment may provide a dichotomous yes/no output as to whether the subject from whom the biological sample was obtained has or does not have GBM, or will or will not develop further disease. As another example, the risk assessment may also provide a risk classification based on the expression levels of various GBM marker genes.

### Obtaining or Storing a Biological Sample

In one embodiment, a biological sample is obtained prior to determining the set of expression profiles. A biological sample may be, for example, a blood sample, preferably, a whole blood sample, or any sample containing peripheral blood cells. For example, a 20-ml non-fasting blood sample may be collected. Blood may be drawn into a 10 ml plain red-top vacutainer and a 10 ml lavender-top vacutainer containing K₃-EDTA (1 mg/ml). Blood in the plain vacutainer may be allowed to clot at ambient temperature and is then centrifuged to recover serum. Serum can be aliquoted and stored at −80° C. until analysis. In one embodiment, a mononuclear blood cell fraction may be isolated from the biological sample. In another embodiment, lymphocytes may be isolated from the biological sample. In another embodiment, a cell fraction enriched for mononuclear blood cells may be obtained from the biological sample. In another embodiment, a cell fraction enriched for lymphocytes may be obtained from the biological sample. For example, the lavender-top vacutainer may be centrifuged at 85 g for 20 minutes at 4° C. to separate the red cells, white cells, and plasma. Fractions may be aliquoted and stored at −80° C. until analysis. Urine samples may also be collected at this time. Samples may be immediately aliquoted and stored at −80° C. until analysis. A biological sample may be, for example, a fluid sample obtained from a biopsy; samples may be immediately aliquoted and stored at −80° C. until analysis.

Samples can be obtained at multiple time points. For example, the samples may be collected on a monthly, bi-monthly, half-yearly, or yearly time points, or any point between. Preferably, the sample is collected at the first sign of symptoms of GBM.

Once a biological sample is obtained, it may then be used to determine a set of expression profiles of GBM marker genes using any of the steps described herein.

### Patient Selection

In one aspect, provided herein is a method of selecting a patient population for prophylactic therapy. Once a biological sample has been assessed using the methods described herein, patients diagnosed as having GBM are selected for prophylactic therapy.

### Therapies

Malign gliomas and glioblastomes in humans are, in most cases, treated by surgical removal of the tumor tissue, followed by chemotherapy and/or irradiation. In many of the cases, the chemotherapy treatment is not satisfactory, since there are two barriers to overcome for cytostatic agents in the treatment step before having full effect, i.e., the blood brain barrier (BBB) and a frequently occurring intrinsic chemoresistance of the blood brain barrier and of the tumor. Hence, it is essential in the course of the pharmacological treatment of brain tumors of the above kind to develop strategies for a transport of said chemotherapeutic agents across the blood brain barrier in vivo. Among the known methods are methods of opening the brain (invasive treatment) for opening the blood brain barrier, methods of modifying the pharmacological agent, whereby the modification enables the agent to pass across the blood brain barrier, and methods of using chimeric peptides for targeting small pharmacologically effective molecules to specific sites within the brain. Other conventional methods of diagnosis are contemplated and included herein.

Provided herein is a method of treating a brain tumor in a subject, comprising: (a) determining the amount of at least one miRNA in cancer cells, relative to control cells; and (b) altering the amount of miRNA expressed in the cancer cells by (i) administering to the subject an effective amount of at least one isolated miRNA, a precursor thereof, an isolated variant thereof, or a biologically active fragment thereof, or (ii) administering to the subject an effective amount of at least one compound for inhibiting expression of the at least one miRNA.

A brain tumor to be treated using methods described herein can be, for example, glioblastoma multiforme. In one aspect, the at least one miRNA detected in a test sample can be expressed in increased levels compared to a control sample. Alternatively, the at least one miRNA detected in a test sample can be expressed in decreased levels compared to a control sample.

In one aspect, provided herein is a method of treating a brain tumor or related disease in a subject, comprising: (a) determining the amount of at least one miRNA selected from the genes in Table 1 in cancer cells, relative to control cells; and (b) altering the amount of miRNA expressed in the cells by (i) administering to the subject an effective amount of at least one isolated miRNA, a precursor thereof, an isolated variant thereof, or a biologically active fragment thereof, or (ii) administering to the subject an effective amount of at least one compound for inhibiting expression of the at least one miRNA.

In another aspect, provided herein is a method of treating a brain tumor or related disease in a subject, comprising: (a) determining the amount of at least one miRNA selected from the genes in Table 2 in cancer cells, relative to control cells; and (b) altering the amount of miRNA expressed in the cells by (i) administering to the subject an effective amount of at least one isolated miRNA, a precursor thereof, an isolated variant thereof, or a biologically active fragment thereof, or (ii) administering to the subject an effective amount of at least one compound for inhibiting expression of the at least one miRNA.

Cancer cells may include cells obtained from a tissue sample containing cancer cells, biopsy fluid, blood or urine. In one non-limiting embodiment, a brain tumor to be treated is glioblastoma multiforme.

In one embodiment, the methods further comprise administering to the subject one or more therapeutic regimens.

In such methods, determining the amount of at least one miRNA selected from the genes in Table 1 in cancer cells, relative to control cells may comprise (i) comparing (a) a set of expression profiles of brain tumor marker genes in a biological sample from the subject, the set comprising expression profiles of a plurality of brain tumor marker genes from Table 1, to (b) a set of expression profiles of brain tumor marker genes in a biological sample from a control subject.

In such methods, determining the amount of at least one miRNA selected from the genes in Table 2 in cancer cells, relative to control cells may comprise (i) comparing (a) a set of expression profiles of brain tumor marker genes in a biological sample from the subject, the set comprising expression profiles of a plurality of brain tumor marker genes from Table 2, to (b) a set of expression profiles of brain tumor marker genes in a biological sample from a control subject.

In one embodiment, the methods further comprise obtaining the set of expression profiles prior to the comparing step.

In one embodiment, at least 30%, of the genes, at least 50% of the genes, at least 75% of the genes, at least 90% of the genes, or any percentage in between are selected from the brain tumor marker genes listed in Table 1.

In one embodiment, at least 30%, of the genes, at least 50% of the genes, at least 75% of the genes, at least 90% of the genes, or any percentage in between are selected from the brain tumor marker genes listed in Table 2.

In one aspect another embodiment, a subject is administered or more therapeutic regimens. In one embodiment, one or more miRNA described herein are administered to a subject having GBM.

Pharmaceuticals either comprising the miRNA described herein or consisting essentially of the miRNA described herein can be directly administered as nucleic acids or administered as part of a viral delivery vehicle or any other suitable carrier. Suitable dosages include for example about 0.1-about 1.0 μg/kg body weight, about 1-about 10 μg/kg of body weight, about 10-about 100 μg/kg of body weight. Other suitable doses include for example about 1-about 10 mg/ml or about 10-about 100 mg/ml. A suitable dose is a therapeutically effective amount of the nucleic acids disclosed herein that reduce tumor growth or shrink preexisting tumors. The compositions can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice. Suitable routes include oral, intraperitoneal, muscular, intramuscular, intravenous, buccal, subcutaneous, sublingual, and topical routes. For injection, the therapeutic compositions can be formulated in liquid solutions, preferably in physiologically compatible buffers such as Hank's solution or Ringer's solution. In addition, the therapeutic compositions may be formulated in solid form and redissolved or suspended immediately prior to use. Lyophilized forms are also included. Pyrogen free forms are also included.

The therapeutic compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Other therapeutic regimens may include, but are not limited to:
(i) Imatinib Mesylate plus hydroxyurea (HU);
(ii) TLN-4601;
(iii) XL184;
(iv) Vorinostat, Isotretinoin and Carboplatin (in combination with surgery as needed);
(v) TransMID™;
(vi) Sutent (Sunitinib, SU11248);
(vii) Cotara (131-labeled Iodine-ch-TNT-1/B Monoclonal antibody);
(viii) dendritic cell based vaccines with autologous dendritic cells and temozolomide in combination with radiotherapy;
(ix) radiation boost comprising temozolomide, brachytherapy and external beam radiation therapy;
(x) radiation therapy and temozolomide followed by temozolomide plus sorafenib;
(xi) IL-13-PE38QQR (surgery and catheter placement; drug prolifespan 20 with carmustine implant (GLIADEL® Wafer); procedure of surgery and wafer placement;
(xii) AQ4N in combination with radiotherapy and temozolomide;
(xiii) CT-322 in combination with Irinotecan hydrochloride;
(xiv) Zarnestra with standard radiation therapy;
(xv) Everolimus (RAD001);
(xvi) Sarasar and Temodar;
(xvii) TP-38;
(xviii) Gliadel followed by Avastin and Irinotecan;
(xix) Erlotinib (Tarceva, OSI,774);
(xx) Chloroquine;
(xxi) Temozolomide and Zarnestra (R115777);
(xxii) 6-TG, Capecitabine and Celecoxib plus temozolomide or CCNU;
(xxiii) MPC-6827 and carboplatin;
(xxiv) Cilengitidine (EMD 121974);
(xxv) ZD1839 (Iressa) and radiotherapy;
(xxvi) Sorafenib plus protracted temozolomide;
(xxvii) CYT997 in combination with carboplatin and etoposide;
(xxviii) Suramin plus radiation therapy;
(xxix) Temozolomide and SCH66336;
(xxx) Photo Radiosurgery System (Intrabeam);
(xxxi) Celecoxib, Isotretinoin, Temozolomide and thalidomide;
(xxxii) AEE788;
(xxxiii) Gliadel Wafer, Temodar and Radiotherapy;
(xxxiv) Cetuximab, Temozolomide and radiation therapy;
(xxxv) Enzastaurin, Temozolomide and radiation;
(xxxvi) Dendritic cell immunotherapy;
(xxxvii) Carmusine and Temozolomide;
(xxxviii) Interleukin 4 PE38KDEL cytotoxin followed by conventional surgery;
(xxxix) Prinomastat and temozolomide;
(xxxx) Gefitinib;
(xxxxi) Leflunomide;
(xxxxii) Procarbazine hydrochloride;
(xxxxiii) Irinotecan hydrochloride, thalidomide following radiation therapy;
(xxxxiv) Celecoxib in combination with radiation therapy; or
(xxxxv) Surgery.

Treatment of cancer with antineoplastic compounds may be accompanied by administration of pharmaceutical agents that can alleviate the side effects produced by the antineoplastic agents. Such agents suitable for use herein include, but are not limited to, anti-emetics, anti-mucositis agents, pain management agents, infection control agents, and anti-anemia/anti-thrombocytopenia agents. Examples of anti-emetics suitable for use herein include, but are not limited to, 5-hydroxytryptamine 3 receptor antagonists, metoclopramide, steroids, lorazepam, ondansetron, cannabinoids, their analogues and derivatives. Examples of anti-mucositis agents suitable for use herein include, but are not limited to, palifermin (keratinocyte growth factor), glucagon-like peptide-2, teduglutide, L-glutamine, amifostin, and fibroblast growth factor 20. Examples of pain management agents suitable for use herein include, but are not limited to, opioids, opiates, and non-steroidal anti-inflammatory compounds. Examples of agents used for control of infection suitable for use herein include, but are not limited to, antibacterials such as aminoglycosides, penicillins, cephalosporins, tetracyclines, clindamycin, lincomycin, macrolides, vancomycin, carbapenems, monobactams, fluoroquinolones, sulfonamides, nitrofurantoins, their analogues and derivatives. Examples of agents that can treat anemia or thrombocytopenia associated with chemotherapy suitable for use herein include, but are not limited to, erythropoietin, and thrombopoietin.

### Diagnosis

The present invention relates generally to detection of nucleic acids, and more specifically to detection of small RNA such as micro-RNA (miRNA).

In one embodiment, the first test to diagnose brain and spinal column tumors is a neurological examination. Special imaging techniques (computed tomography, and magnetic resonance imaging, positron emission tomography) are also employed. Laboratory tests include the EEG and the spinal tap. A biopsy, a surgical procedure in which a sample of tissue is taken from a suspected tumor, helps doctors diagnose the type of tumor.

MiRNA molecules described herein can be used to diagnose whether a subject has, or is at risk for developing, a brain tumor or a related disease such as, for example, glioblastoma multiforme.

Provided herein is a method of diagnosing whether a subject has, or is at risk for developing, a brain tumor or a related disease, comprising measuring the level of at least one miRNA selected from Table 1 in a test sample, relative to the level of a corresponding miRNA in a control sample, is indicative of the subject either having, or being at risk for developing a brain tumor or a related disease.

Provided herein is a method of diagnosing whether a subject has, or is at risk for developing, a brain tumor or a related disease, comprising measuring the level of at least one miRNA selected from Table 2 in a test sample, relative to the level of a corresponding miRNA in a control sample, is indicative of the subject either having, or being at risk for developing a brain tumor or a related disease.

A brain tumor to be diagnosed using methods described herein can be, for example, glioblastoma multiforme. In one aspect, the at least one miRNA detected in a test sample can be expressed in increased levels compared to a control sample. Alternatively, the at least one miRNA detected in a test sample can be expressed in decreased levels compared to a control sample.

### Kits and Arrays

Provided herein are kits and arrays for use for identifying a subject having, or at risk of having a brain tumor or a related disease such as, for example, GBM.

In one aspect of the present invention are kits for use in the methods for identifying a subject having, or at risk of having GBM, comprising: (i) a set of nucleic acid probes that hybridize under high stringency conditions to the nucleotide sequences of one to twenty genes in a biological sample comprising cells from a subject, wherein at least 20% of the genes are selected from the GBM marker genes listed in Table 1, for determining the expression profiles of said genes; and an insert describing: (a) an expression profile of one or more of the GBM marker genes in blood samples from one or more subjects who do not have GBM; (b) an expression profile of one or more GBM marker genes in blood samples from one or more subjects who have GBM; or (c) a multimarker classifier, wherein the multimarker classifier was obtained by a comparison of expression levels of the GBM marker genes in a plurality of subjects who do not have GBM to expression levels of the GBM marker genes in a plurality of subjects who do have GBM.

In one embodiment of the kits, the set of nucleic acid probes comprise primers for RT-PCR amplification of the mRNAs for the one to sixty marker genes of Table 1.

In yet another aspect of the present invention are kits for use in the methods for identifying a subject having, or at risk of having GBM, comprising: (i) a set of nucleic acid probes that hybridize under high stringency conditions to the nucleotide sequences of one to twenty genes in a biological sample comprising cells from a subject, wherein at least 20% of the genes are selected from the GBM marker genes listed in Table 2, for determining the expression profiles of said genes; and an insert describing: (a) an expression profile of one or more of the GBM marker genes in blood samples from one or more subjects who do not have GBM; (b) an expression profile of one or more GBM marker genes in blood samples from one or more subjects who have GBM; or (c) a multimarker classifier, wherein the multimarker classifier was obtained by a comparison of expression levels of the GBM marker genes in a plurality of subjects who do not have GBM to expression levels of the GBM marker genes in a plurality of subjects who do have GBM.

In one embodiment of the kits, the set of nucleic acid probes comprise primers for RT-PCR amplification of the mRNAs for the one to five marker genes of Table 2.

In yet another aspect of the present invention are nucleic acid arrays comprising nucleic acid probes that hybridize under high stringency conditions to the nucleotide sequences of no more than one to sixty genes, wherein at least 20% of the genes are selected from the GBM marker genes listed in Table 1.

In yet another aspect of the present invention are nucleic acid arrays comprising nucleic acid probes that hybridize under high stringency conditions to the nucleotide sequences of no more than one to five genes, wherein at least 20% of the genes are selected from the GBM marker genes listed in Table 2.

In one embodiment of the nucleic acid arrays, the nucleic acid array is provided as one or more multiwell plates, comprising primers for RT-PCR amplification of the mRNAs for the one to twenty GBM marker genes.

In another embodiment of the nucleic acid arrays, the nucleic acid array is provided as a nucleic acid hybridization microarray.

In another embodiment of the nucleic acid arrays, at least 10% of the genes of the genes are selected from the GBM marker genes listed in Table 1.

In another embodiment of the nucleic acid arrays, at least 10% of the genes of the genes are selected from the GBM marker genes listed in Table 2.

In another embodiment of the nucleic acid arrays, at least 30% of the genes of the genes are selected from the GBM marker genes listed in Table 1.

In another embodiment of the nucleic acid arrays, at least 30% of the genes of the genes are selected from the GBM marker genes listed in Table 2.

In another embodiment of the nucleic acid arrays, at least 50% of the genes of the genes are selected from the GBM marker genes listed in Table 1.

In another embodiment of the nucleic acid arrays, at least 50% of the genes of the genes are selected from the GBM marker genes listed in Table 2.

In another embodiment of the nucleic acid arrays, at least 90% of the genes of the genes are selected from the GBM marker genes listed in Table 1.

In another embodiment of the nucleic acid arrays, at least 90% of the genes of the genes are selected from the GBM marker genes listed in Table 2.

In another embodiment of the nucleic acid arrays, the array comprises nucleic acid probes that hybridize under high stringency conditions to the nucleotide sequences of no more than three to twenty genes.

In another embodiment of the nucleic acid arrays, the array comprises nucleic acid probes that hybridize under high stringency conditions to the nucleotide sequences of no more than five to fifteen genes.

In another embodiment of the nucleic acid arrays, the array comprises nucleic acid probes that hybridize under high stringency conditions to the nucleotide sequences of no more than seven to twelve genes.

### EXAMPLES

The following specific examples are illustrative, but do not limit the remainder of the disclosure of the invention in any way whatsoever.

### Sample Collection

Tumors are classified according to the World Health Organization criteria into the various subtypes of low-grade astrocytomas, anaplastic astrocytomas and glioblastoma multiforme. Low-grade gliomas (grade II), anaplastic astrocytomas (grade III) and glioblastoma multiforme (GBM, grade IV) are characterized by a different profile of mutations and genetic alterations. Histopathological diagnoses are made according to the World Health Organization guidelines and evaluated in formalin-fixed paraffin-embedded hematoxylin/eosin-stained tissue slices. Tumors are collected from patients operated on at a hospital. Fresh tissue is frozen immediately following surgery in liquid nitrogen and stored at −70° C. until processing. Sample collection and processing are performed according to the regulations of the committee on research involving human subjects of the Organization Institutional Review Board (IRB).

### Whole Blood Collection and Isolation of RNA.

PAXgene™ Blood RNA tubes and Blood RNA Kit (PreAnalytiX, Qiagen, Inc) are used for collection of whole blood (5 ml) and stabilization, purification, and isolation of RNA. Total mRNA is isolated from whole blood samples using the PAXgene Blood RNA Kit (Qiagen Inc., Valencia, Calif.) following standard procedures. Total RNA concentrations are calculated by determining absorbance at 260 nm (Spectramax Plus 384 spectrophotometer, Molecular Devices, Sunnyvale, Calif.) in 10 mM Tris-HCl. Protein contamination is monitored using the A260/A280 ratio. To assure high quality, all samples have an A260/A280 ratio of >1.8. The GLOBINclear kit (Ambion, Austin, Tex.) is used to decrease the masking effect abundant globin mRNA has on less abundant mRNA. Purified RNA samples are used to perform microarray experiments or immediately stored frozen in a buffer at −80° C. for qRT-PCR experiments designed to verify microarray results.

Samples are assessed for quality control and fluorescently labeled. Quality control of total RNA is analyzed using an Agilent 2100 Bioanalyzer capillary electrophoresis system, and spectrophotometric scan of each sample is conducted in the UV range from 220-300 nm. Those RNA samples that pass QC are amplified using Ambion's MessageAmp I kit and the subsequent RNA labeled with a fluorescent dye tag. RNA samples, including reference RNAs, are QC'ed, amplified, and labeled using standardized protocols.

Commercially printed microarrays having the 4×44k slide format (probes are 60-mers and the array format is two-channel) from Agilent Technologies (Santa Clara, Calif.) are used. Array information is obtained from RefSeq, Goldenpath Ensembl Unigene Human Genome (Build 33) and GenBank. Array processing protocols (i.e., hybridization and washes) are fully automated with the use of two Robbins Scientific Hybridization Incubator equipped with Agilent Technologies rotisserie assemblies. Protocols and reagents used are outlined at www.chem.agilent.com/Scripts/PDS.asp?lPage=34519. Post-hybridized arrays are imaged using an Agilent Technologies DNA Microarray Scanner.

Array images may be quantified, tested for signal quality and normalized using Agilent Feature Extraction Software v9.5.3 (Agilent Technologies). Statistical data analysis and data visualization are performed using GeneSpring 7.0 microarray analysis software (Agilent Technologies and open-source tools such as those provided by the BioConductor Bioinformatics Resource (www.bioconductor.org/).

Verification of expression data obtained from genomic microarrays is performed using qRT-PCR-based analyses for up to 20 genes identified as classifiers of GBM. First strand cDNA is synthesized by using the High Capacity cDNA Archive Kit (Applied Biosystems, Foster City, Calif.). The reverse transcription reaction for each sample is performed either the day of or the day before the PCR reaction. This is so that cDNA will not be degraded by storage. Testing in our lab has shown that overnight storage of cDNA at 4° C. has negligible effects on PCR results. qRT-PCR is performed in duplicate on 25 μL mixtures, containing 25-150 ng of template cDNA, 12.5 μL of 2×TaqMan® Universal Master Mix (Applied Biosystems), and 1.25 μL of TaqMan® Gene Expression Assay for the gene of interest or control gene (Applied Biosystems, Inc.). Assays that are reported by Applied Biosystems, Inc., (or the appropriate primer-probe set) to pick up genomic DNA are additionally tested for genomic DNA contamination by running a reverse transcriptase minus (RT−) control for every sample. Reactions are run in 96-well plates with optical covers (Applied Biosystems, Inc.) on an ABI PRISM 7000 Real Time PCR machine (Applied Biosystems, Inc.) using the default cycling conditions. Four point control cDNA is used for primer efficiency comparison of all Assays on Demand based on the slope of each standard curve calculated by the ABI PRISM 7000 SDS Software, Version 1.1.

### Statistical and Bioinformatics Analysis

Analysis is conducted using natural log-transformed data. Both supervised and unsupervised approaches are used to identify inherent differences in gene expression patterns between GBM cases and controls. Unsupervised methods, such as cluster or principal component analysis (PCA), are commonly used in microarray analyses. PCA is used to reduce the high dimension microarray data to 2 or 3 dimensions for easy visualization thus allowing similar comparisons across samples. Cluster analyses simultaneously group samples and genes that share similar expression patterns. The color representation of heat mapping from cluster analysis reveals unique gene signatures to distinguish various sub-groups of participants in a global genomic fashion. Cluster and TreeView software is used to construct a phylogenetic tree of genes (that are differentially expressed). The programs use a hierarchical clustering algorithm that utilizes the Pearson's correlation coefficient.

Although unsupervised methods provide the means to visualize global gene expression patterns, it is more appropriate to use supervised approaches to identify subset of genes that can robustly distinguish GBM cases from controls. The support vector machine (SVM), the significance analysis of microarrays (SAM), and the Shrunken Centroids methods are three candidate methods that are widely used to classify disease status. Permutations are also used to estimate the percentage of genes identified by chance, false discovery rate (FDR), for genes with scores greater than an adjustable threshold. The FDR, q-value of a selected gene corresponds to the FDR for the gene list that includes the gene and all genes that were more significant. Some investigators use a direct approach to gene selection to build classifiers using a subset of genes in a SVM model. This is done by choosing K genes with the largest absolute value of scores in an SVM model built using the RankGene system. The system takes into account several criteria such as t-test statistic, information gain, and variance of expression to determine the discriminative strength of individual genes. Although the aforementioned approaches are computationally straight-forward, investigators have noted that the possibility of collinearity among the selected K genes may reduce classifier performance; other analytical approaches to gene selection are also used. Data is analyzed using other methods such as greedy forward selection, genetic algorithms, and gradient-based leave-one-out gene selection (GLGS) algorithms. We recognize that use of multiple approaches will yield different results, so we have defined a priori our preferred criteria for classifier gene selection. Genes that satisfy the following three criteria in comparisons between GBM cases and controls constitute the final set of genes: (1) Student's t-test p-value<0.001; (2) fold change differences≧2.0; and (3), false discovery rates (FDR)≦10% as using (SAM). We also follow the standards advocated by the Karnofsky Performance Score.

The “10-fold cross validation” approach is used on the derivation data set to evaluate the performance of classifiers identified. The derivation data is divided into 10 equal parts, each with 12 samples. Eleven (11) parts of the data are selected as a “test or training set” from which a classification model with K gene will be constructed to confirm its prediction performance on the remaining excluded part. The decision call for each excluded sample tested is made based on the prediction function/score provided by each method. For instance the Shrunken Centroids methods provide a predictive probability of being in the GBM. The procedure is repeated 12 times then the overall error rate will be estimated. The overall error depends on the number of K genes in the model. Hence, the number is varied by changing the tuning parameter when using the Shrunken Centroids method. The optimal number of genes, K, or equivalently the optimal tuning parameter is chosen such that the overall error rater reaches its minimum. Permutation testing is used to assess the significance of the observed error rate. Briefly, 60 samples are randomly relabeled as belonging to the GBM group and the remaining 60 in the term control group. Then the same 10-fold cross validation analysis as previously described is conducted, and overall error rates recorded based on the optimal K genes from this permuted data. This procedure is repeated 1,000 times to obtain a null distribution of the overall error rate, allowing us to measure the significance of overall error rates in the derivation set with correct classification. Exploratory analyses are conducted for estimating error rates. Methods that trade off bias for low variance, such as balance bootstrap re-sampling approaches, which have been shown to be a variance reducing technique, are used.

Microarray findings are confirmed using qRT-PCR methods. qRT-PCR for up to 20 genes, is performed on all 240 samples in both the derivation and the validation set. Correlation coefficients (e.g., Spearman's correlation coefficients) of expression values from microarray and qRT-PCR approaches are assessed.

The observed error rate for the 120 samples in the validation data set is calculated based on the classifier constructed from the 120 independent samples from the derivation data set. GBM status label on the derivation set is permuted to obtain a null classifier and validate its prediction performance on the validation data set. This procedure is repeated 1,000 times, and significance levels of the observed error rates obtained. As an alternative means of testing classification accuracy, exploratory methods such as PCA and multi-dimensional scaling (MDS) are also used. A 2 (GBM versus control) of the 120 validation samples based on the K genes in the classifier constructed from the derivation set is constructed.

Bioinformatics approaches are used to retrieve and interpret complex biological interactions. Two independent tools are used: (1) DAVID and (2) Ingenuity Pathway Analysis (IPA) software (Ingenuity, Redwood City, Calif.) to study systems biology and to explore mechanistic hypotheses. In analysis based on DAVID, a comprehensive set of functional annotation tools and an enrichment analytic algorithm technique are used to identify enriched functional-related gene groups. A modified Fisher Exact p-value, an EASE score, are used to measure the gene-enrichment in annotation terms by comparing the proportion of genes that fall under each category or term to the human genome background. An overall enrichment score for the group is derived as the geometric mean (in log scale) of member' p-values (EASE score) in a corresponding annotation cluster. In IPA, Ingenuity Pathways Knowledge Base (IPKB), a published and peer-reviewed database and computational algorithms is used to identify local networks that are particularly enriched for the Network Eligible Genes, defined as genes in our list of differentially expressed genes with at least one previously defined connection to another gene in the IPKB. A score, that takes into account the number of Network Eligible Genes and the size of the networks, is calculated using a Fisher Exact test as the negative log of the probability that the genes within that network are associated by chance. A score of 3 (p-value corresponding to 0.001) as the cutoff for significance of the network is used. The overall enrichment score in the analysis conducted using DAVID and the network score obtained in IPA is used to rank the biological significance of gene function clusters and networks, respectively, in GBM.

### MicroRNA Sequencing:

MicroRNAs were isolated and sequenced using the next generation sequencing technology developed by Solexa (now Illumina, Inc.). MicroRNAs from normal brain tissues and GBM tumors were isolated and a microRNA library constructed using Illumina's DGE-small RNA sample preparation kit (Catalogue FC-102-1009). In brief, RNAs were purified by polyacrylamide gel electrophoresis (PAGE) to isolate microRNAs in the range of 18-30 nucleotides (nt), and ligated with proprietary adapters to the 5′ and 3′ termini of the RNA. The samples were used as templates for cDNA synthesis. The cDNA was amplified with 18 PCR cycles to produce sequencing libraries that were subjected to Solexa's proprietary sequencing-by-synthesis method.

### Analysis of MicroRNA Sequencing Data:

Individual sequence reads with the base quality scores were produced by Illumina/Solexa. Identification of known microRNAs was performed by BLAST search against the miRNA database—miRBase (microrna.sanger.ac.uk/) allowing no matches. The read counts for miRNAs were tabulated and normalized to counts per million (tpm). For comparison between the normal brain tissues and the brain cancer tissues, total sequence tag counts were used for normalization.

### TaqMan® MicroRNA Assays

Biological samples were obtained from the brain tissue bank at Swedish Medical Center/Neuroscience Institute.

Differential gene expression of miRNAs between GBM and normal brain tissues were assessed utilizing the TaqMan® MicroRNA Assay (Applied Biosystems, Inc.) according to manufacturer's directions. MicroRNA was normalized to control U6RNA. RNU6B is the control (CGCAAGGAUGACACGCAAAUUCGUGAAGCGUUCCAUAUUUUU; SEQ ID NO: 61).

A number of miRNA analyzed were found to be expressed in increased levels compared to normal signature counts whereas others were decreased compared to normal signature counts (See Table 3).

TABLE 3
Differentially expressed miRNAs identified by Solexa's SBS sequencing

| | | GBM | Normal | |
|---|---|---|---|---|
| | | signature | signature | GBM/Normal |
| id | sequences | counts# | counts | Ratio |
| hsa-miR-15b | UAGCAGCACAUCAUGGUUUACA | 839 | 18 | 46.40 |
| hsa-miR-23a | AUCACAUUGCCAGGGAUUUCC | 2376 | 145 | 16.43 |
| hsa-miR-146b | UGAGAACUGAAUUCCAUAGGCU | 8914 | 885 | 10.07 |
| hsa-miR-23b | AUCACAUUGCCAGGGAUUACC | 466 | 36 | 12.89 |
| hsa-miR-130a | CAGUGCAAUGUUAAAAGGGCAU | 388 | 72 | 5.37 |
| hsa-miR-199a | ACAGUAGUCUGCACAUUGGUUA | 2500 | 307 | 8.14 |
| hsa-miR-191 | CAACGGAAUCCCAAAAGCAGCUG | 2780 | 831 | 3.34 |
| hsa-miR-25* | CAUUGCACUUGUCUCGGUCUGA | 233 | 54 | 4.30 |
| hsa-miR-1 | UGGAAUGUAAAGAAGUAUGUAU | 528 | 2060 | 0.26 |
| hsa-miR-9 | UCUUUGGUUAUCUAGCUGUAUGA | 1367 | 11782 | 0.12 |
| hsa-miR-10a | UACCCUGUAGAUCCGAAUUUGUG | 62 | #VALUE! | #VALUE! |
| hsa-miR-629 | UGGGUUUACGUUGGGAGAACU | 62 | #VALUE! | #VALUE! |
| hsa-miR-1262 | AUGGGUGAAUUUGUAGAAGGAU | 47 | #VALUE! | #VALUE! |
| hsa-miR-452 | AACUGUUUGCAGAGGAAACUGA | 47 | #VALUE! | #VALUE! |
| hsa-miR-1827 | UGAGGCAGUAGAUUGAAU | 47 | #VALUE! | #VALUE! |
| hsa-miR-628-5p | AUGCUGACAUAUUUACUAGAGG | 47 | #VALUE! | #VALUE! |
| hsa-miR-181a-2* | AACAUUCAACGCUGUCGGUGAGU | 62 | 54 | 1.15 |
| miR-127-3p | UCGGAUCCGUCUGAGCUUGGCU | 233 | 2223 | 0.10 |
| hsa-miR-433 | AUCAUGAUGGGCUCCUCGGUGU | 16 | 632 | 0.02 |
| hsa-miR-219 | UGAUUGUCCAAACGCAAUUCU | 16 | 1373 | 0.01 |
| hsa-miR-886-5p | CGGGUCGGAGUUAGCUCAAGCGG | 342 | #VALUE! | #VALUE! |
| hsa-miR-21* | UAGCUUAUCAGACUGAUGUUGA | 388 | #VALUE! | #VALUE! |
| hsa-miR-21 | UAGCUUAUCAGACUGAUGUUGA | 198194 | 3289 | 60.26 |
| hsa-miR-142-5p | CAUAAAGUAGAAAGCACUACU | 512 | 18 | 28.36 |
| hsa-miR-92b* | UAUUGCACUCGUCCCGGCCUCC | 497 | 18 | 27.50 |
| hsa-miR-15a | UAGCAGCACAUAAUGGUUUGUG | 248 | 18 | 13.75 |
| hsa-miR-106b | UAAAGUGCUGACAGUGCAGAU | 699 | 54 | 12.89 |
| hsa-miR-27a | UUCACAGUGGCUAAGUUCCGC | 202 | 18 | 11.17 |
| hsa-miR-365 | UAAUGCCCCUAAAAAUCCUUAU | 171 | 18 | 9.45 |
| hsa-miR-143* | UGAGAUGAAGCACUGUAGCUC | 171 | 18 | 9.45 |
| hsa-miR-186 | CAAAGAAUUCUCCUUUUGGGCU | 637 | 72 | 8.81 |
| hsa-miR-34a | UGGCAGUGUCUUAGCUGGUUGU | 155 | 18 | 8.59 |
| hsa-miR-199b-3p | ACAGUAGUCUGCACAUUGGUUA | 2500 | 307 | 8.14 |
| hsa-miR-1274b | UCCCUGUUCGGGCGCCA | 140 | 18 | 7.73 |
| hsa-miR-34c-5p | AGGCAGUGUAGUUAGCUGAUUGC | 9022 | 1193 | 7.56 |
| hsa-miR-223* | UGUCAGUUUGUCAAAUACCCCA | 124 | 18 | 6.87 |
| hsa-miR-204 | UUCCCUUUGUCAUCCUAUGCCU | 109 | 18 | 6.02 |
| hsa-miR-99b* | CACCCGUAGAACCGACCUUGCG | 109 | 18 | 6.02 |
| hsa-miR-31 | AGGCAAGAUGCUGGCAUAGCU | 311 | 54 | 5.73 |
| hsa-miR-532-5p | CAUGCCUUGAGUGUAGGACCGU | 311 | 54 | 5.73 |
| hsa-miR-1308 | GCAUGGGUGGUUCAGUGG | 8541 | 1626 | 5.25 |
| hsa-miR-210 | CUGUGCGUGUGACAGCGGCUGA | 93 | 18 | 5.16 |
| hsa-miR-16 | UAGCAGCACGUAAAUAUUGGCG | 854 | 181 | 4.73 |
| hsa-miR-126 | UCGUACCGUGAGUAAUAAUGCG | 78 | 18 | 4.30 |
| hsa-miR-24 | UGGCUCAGUUCAGCAGGAACAG | 3742 | 922 | 4.06 |
| hsa-miR-598 | UACGUCAUCGUUGUCAUCGUCA | 109 | 488 | 0.22 |
| hsa-miR-374b | AUAUAAUACAACCUGCUAAGUG | 31 | 145 | 0.21 |
| hsa-miR-184 | UGGACGGAGAACUGAUAAGGGU | 31 | 145 | 0.21 |
| hsa-miR-330-3p | GCAAAGCACACGGCCUGCAGAGA | 109 | 560 | 0.19 |
| hsa-miR-9* | UCUUUGGUUAUCUAGCUGUAUGA | 78 | 434 | 0.18 |
| hsa-miR-760 | CGGCUCUGGGUCUGUGGGGA | 16 | 126 | 0.12 |
| hsa-miR-379 | UGGUAGACUAUGGAACGUAGG | 16 | 126 | 0.12 |
| hsa-miR-7 | UGGAAGACUAGUGAUUUUGUUGU | 792 | 7463 | 0.11 |
| hsa-miR-128 | UCACAGUGAACCGGUCUCUUU | 419 | 4301 | 0.10 |
| hsa-miR-543 | AAACAUUCGCGGUGCACUUCUU | 16 | 181 | 0.09 |
| hsa-miR-485-5p | AGAGGCUGGCCGUGAUGAAUUC | 31 | 379 | 0.08 |
| hsa-miR-499-5p | UUAAGACUUGCAGUGAUGUUU | 16 | 235 | 0.07 |
| hsa-miR-129-3p | AAGCCCUUACCCCAAAAAGCAU | 31 | 488 | 0.06 |
| hsa-miR-124 | UAAGGCACGCGGUGAAUGCC | 62 | 4518 | 0.01 |
| hsa-miR-219-2-3p | AGAAUUGUGGCUGGACAUCUGU | 62 | 4536 | 0.01 |

*normalized to tpm. Only shows those >4 folds, and >10 tpm in at least one sample.

Analysis of hsa-mir-433

Biological samples were obtained from the brain tissue bank at Swedish Medical Center/Neuroscience Institute. TaqMan® MicroRNA Assay for hsa-mir-433 (AB assay ID 001028) (Applied Biosystems) with the target Sequence AUCAUGAUGGGCUCCUCGGUGU (SEQ ID NO: 62) was used.

Quantitative PCR analysis was conducted on the samples as follows:

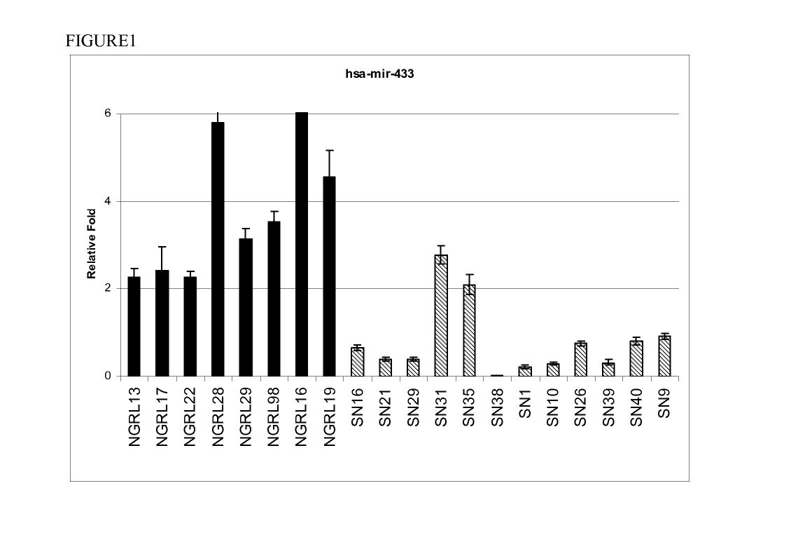

Differential gene expression was observed: Black shaded columns represent normal brain tissues (NGRL series) and hatched columns represent brain cancer samples (SN series). The expression of has-mir-433 was down-regulated in GBM samples (P=0.01, T-test) (FIG. 1).

Quantitative PCR Analysis of hsa-mir-15b

Biological samples were obtained from the brain tissue bank at Swedish Medical Center/Neuroscience Institute. TaqMan® MicroRNA Assay for hsa-mir-15b (AB assay ID 000390) (Applied Biosytems) with the target Sequence UAGCAGCACAUCAUGGUUUACA was used.

Quantitative PCR analysis was conducted on the samples as follows:

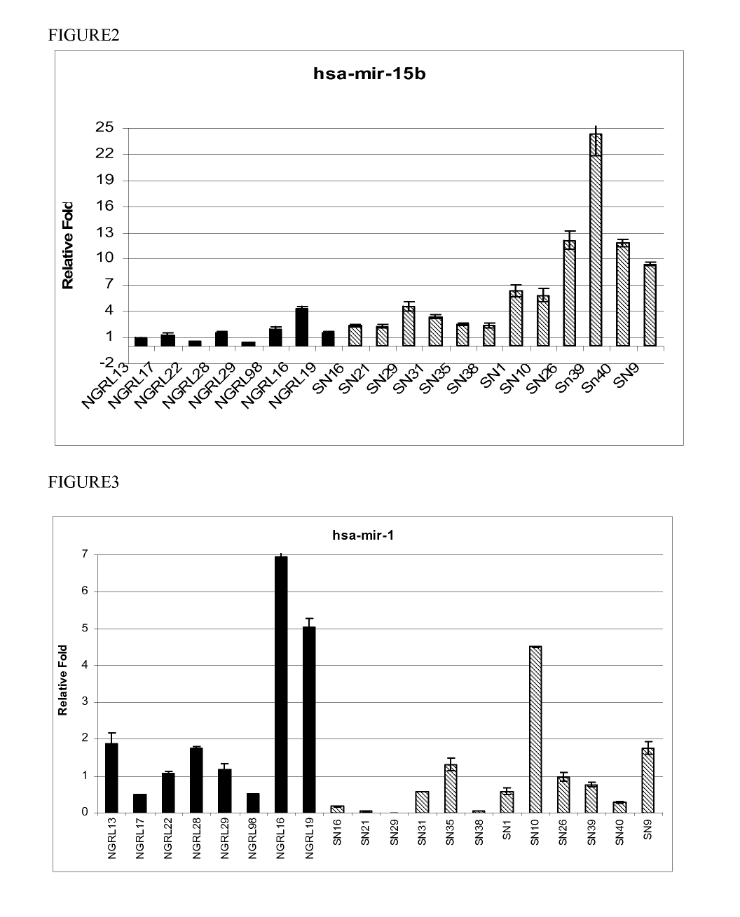

Differential gene expression was observed: black shaded columns represent normal brain tissues (NGRL series) and hatched columns represent brain cancer samples (SN series). The expression of has-mir-15b was slightly up-regulated in GBM samples (P=0.0034, T-test) (FIG. 2).

Quantitative PCR Analysis of hsa-mir-1

Biological samples were obtained from the brain tissue bank at Swedish Medical Center/Neuroscience Institute. TaqMan® MicroRNA Assay for hsa-mir-1 (AB assay ID 002222) (Applied Biosytems) with the target Sequence UGGAAUGUAAAGAAGUAUGUAU was used.

Quantitative PCR analysis was conducted on the samples as follows:

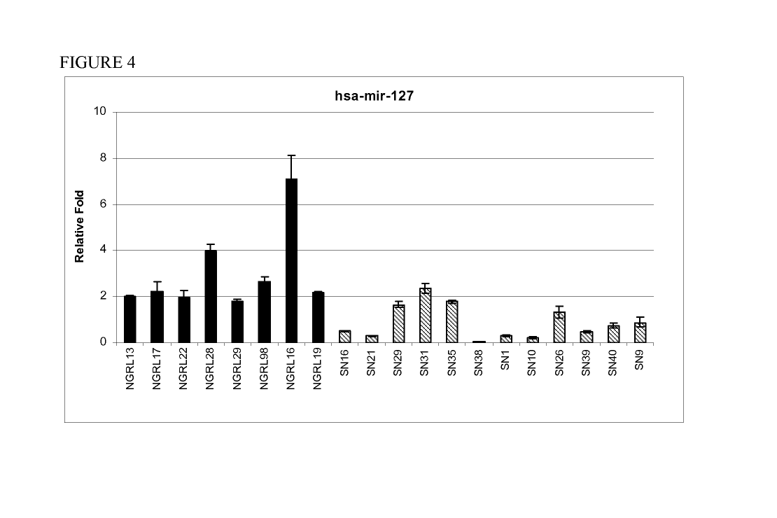

Differential gene expression was observed: black shaded columns represent normal brain tissues (NGRL series) and hatched columns represent brain cancer samples (SN series). The expression of has-mir-1 was down-regulated in GBM samples (P=0.03, T-test) (FIG. 3).

Quantitative PCR Analysis of hsa-mir-127-3p

Biological samples were obtained from the brain tissue bank at Swedish Medical Center/Neuroscience Institute. TaqMan0 MicroRNA Assay for hsa-mir-127-3p (AB assay ID 000452) (Applied Biosytems) with the target Sequence UCGGAUCCGUCUGAGCUUGGCU was used.

Quantitative PCR analysis was conducted on the samples as follows:

Differential gene expression was observed: black shaded columns represent normal brain tissues (NGRL series) and hatched columns represent brain cancer samples (SN series). The expression of has-mir-127-3p was down-regulated in GBM samples (P=0.02, T-test) (FIG. 4).

Quantitative PCR Analysis of hsa-mir-124

Biological samples were obtained from the brain tissue bank at Swedish Medical Center/Neuroscience Institute. TaqMan® MicroRNA Assay for hsa-mir-124 (AB assay ID 001182) (Applied Biosytems) with the target Sequence UAAGGCACGCGGUGAAUGCC was used.

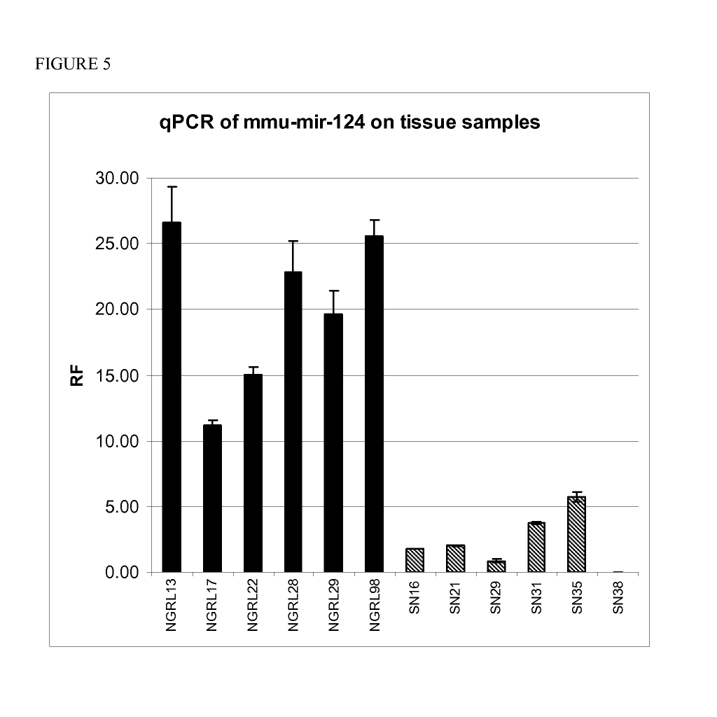

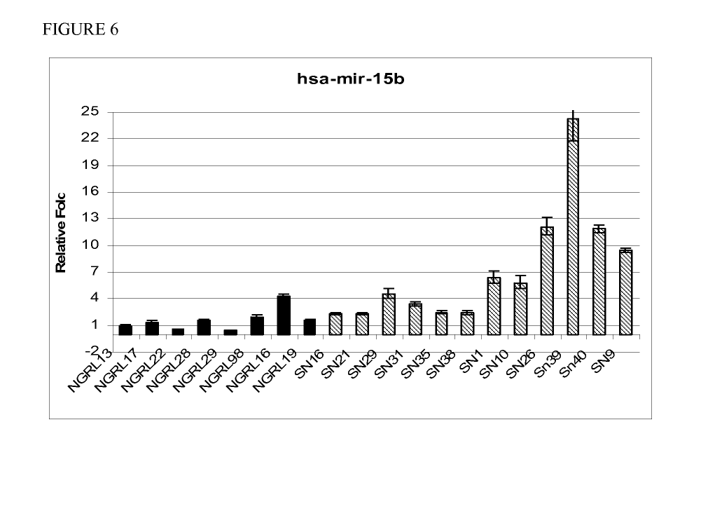

Quantitative PCR analysis was conducted on the samples as follows: Differential gene expression was observed: black shaded columns represent normal brain tissues (NGRL series) and hatched columns represent brain cancer samples (SN series). The expression of has-mir-124 was down-regulated in GBM samples (P=4.58E-04, T-test) (FIG. 5).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Sequence Table

| | | SEQ |
|---|---|---|
| | Sequence | ID NO |
| hsa-miR-15b | UAGCAGCACAUCAUGGUUUACA | 1. |
| hsa-miR-23a | AUCACAUUGCCAGGGAUUUCC | 2. |
| hsa-miR-146b | UGAGAACUGAAUUCCAUAGGCU | 3. |
| hsa-miR-23b | AUCACAUUGCCAGGGAUUACC | 4. |
| hsa-miR-130a | CAGUGCAAUGUUAAAAGGGCAU | 5. |
| hsa-miR-199a | ACAGUAGUCUGCACAUUGGUUA | 6. |
| hsa-miR-191 | CAACGGAAUCCCAAAAGCAGCUG | 7. |
| hsa-miR-25* | CAUUGCACUUGUCUCGGUCUGA | 8. |
| hsa-miR-1 | UGGAAUGUAAAGAAGUAUGUAU | 9. |
| hsa-miR-9 | UCUUUGGUUAUCUAGCUGUAUGA | 10. |
| hsa-miR-10a | UACCCUGUAGAUCCGAAUUUGUG | 11. |
| hsa-miR-629 | UGGGUUUACGUUGGGAGAACU | 12. |
| hsa-miR-1262 | AUGGGUGAAUUUGUAGAAGGAU | 13. |
| hsa-miR-452 | AACUGUUUGCAGAGGAAACUGA | 14. |
| hsa-miR-1827 | UGAGGCAGUAGAUUGAAU | 15. |
| hsa-miR-628-5p | AUGCUGACAUAUUUACUAGAGG | 16. |
| hsa-miR-181a-2* | AACAUUCAACGCUGUCGGUGAGU | 17. |
| miR-127-3p | UCGGAUCCGUCUGAGCUUGGCU | 18. |
| hsa-miR-433 | AUCAUGAUGGGCUCCUCGGUGU | 19. |
| hsa-miR-219 | UGAUUGUCCAAACGCAAUUCU | 20. |
| hsa-miR-886-5p | CGGGUCGGAGUUAGCUCAAGCGG | 21. |
| hsa-miR-21* | UAGCUUAUCAGACUGAUGUUGA | 22. |
| hsa-miR-21 | UAGCUUAUCAGACUGAUGUUGA | 23. |
| hsa-miR-142-5p | CAUAAAGUAGAAAGCACUACU | 24. |
| hsa-miR-92b* | UAUUGCACUCGUCCCGGCCUCC | 25. |
| hsa-miR-15a | UAGCAGCACAUAAUGGUUUGUG | 26. |
| hsa-miR-106b | UAAAGUGCUGACAGUGCAGAU | 27. |
| hsa-miR-27a | UUCACAGUGGCUAAGUUCCGC | 28. |
| hsa-miR-365 | UAAUGCCCCUAAAAAUCCUUAU | 29. |
| hsa-miR-143* | UGAGAUGAAGCACUGUAGCUC | 30. |
| hsa-miR-186 | CAAAGAAUUCUCCUUUUGGGCU | 31. |
| hsa-miR-34a | UGGCAGUGUCUUAGCUGGUUGU | 32. |
| hsa-miR-199b-3p | ACAGUAGUCUGCACAUUGGUUA | 33. |
| hsa-miR-1274b | UCCCUGUUCGGGCGCCA | 34. |
| hsa-miR-34c-5p | AGGCAGUGUAGUUAGCUGAUUGC | 35. |
| hsa-miR-223* | UGUCAGUUUGUCAAAUACCCCA | 36. |
| hsa-miR-204 | UUCCCUUUGUCAUCCUAUGCCU | 37. |
| hsa-miR-99b* | CACCCGUAGAACCGACCUUGCG | 38. |
| hsa-miR-31 | AGGCAAGAUGCUGGCAUAGCU | 39. |
| hsa-miR-532-5p | CAUGCCUUGAGUGUAGGACCGU | 40. |
| hsa-miR-1308 | GCAUGGGUGGUUCAGUGG | 41. |
| hsa-miR-210 | CUGUGCGUGUGACAGCGGCUGA | 42. |
| hsa-miR-16 | UAGCAGCACGUAAAUAUUGGCG | 43. |
| hsa-miR-126 | UCGUACCGUGAGUAAUAAUGCG | 44. |
| hsa-miR-24 | UGGCUCAGUUCAGCAGGAACAG | 45. |
| hsa-miR-598 | UACGUCAUCGUUGUCAUCGUCA | 46. |
| hsa-miR-374b | AUAUAAUACAACCUGCUAAGUG | 47. |
| hsa-miR-184 | UGGACGGAGAACUGAUAAGGGU | 48. |
| hsa-miR-330-3p | GCAAAGCACACGGCCUGCAGAGA | 49. |
| hsa-miR-9* | UCUUUGGUUAUCUAGCUGUAUGA | 50. |
| hsa-miR-760 | CGGCUCUGGGUCUGUGGGGA | 51. |
| hsa-miR-379 | UGGUAGACUAUGGAACGUAGG | 52. |
| hsa-miR-7 | UGGAAGACUAGUGAUUUUGUUGU | 53. |
| hsa-miR-128 | UCACAGUGAACCGGUCUCUUU | 54. |
| hsa-miR-543 | AAACAUUCGCGGUGCACUUCUU | 55. |
| hsa-miR-485-5p | AGAGGCUGGCCGUGAUGAAUUC | 56. |
| hsa-miR-499-5p | UUAAGACUUGCAGUGAUGUUU | 57. |
| hsa-miR-129-3p | AAGCCCUUACCCCAAAAAGCAU | 58. |
| hsa-miR-124 | UAAGGCACGCGGUGAAUGCC | 59. |
| hsa-miR-219-2-3p | AGAAUUGUGGCUGGACAUCUGU | 60. |
| | CGCAAGGAUGACACGCAAAUUC | 61. |
| | GUGAAGCGUUCCAUAUUUUU | |
| | AUCAUGAUGGGCUCCUCGGUGU | 62. |

What is claimed is:

1. A method of diagnosing whether a subject has, or is at risk for developing, a brain tumor, comprising:
(i) obtaining from a biological sample from the subject a set of expression profiles of a plurality of brain tumor marker genes from Table 1;
(ii) comparing (a) the set of expression profiles of brain tumor marker genes in a biological, sample from the subject, the set comprising expression profiles of a plurality of brain tumor marker genes from Table 1, to (b) a set of expression profiles of brain tumor marker genes in a biological sample from a control subject; and
(iii) providing a diagnosis for, or a risk assessment of, a brain tumor based on the comparison.

2. A method for identifying a subject having a brain tumor, comprising determining expression profiles of no more than five to five hundred genes in a biological sample comprising cells from a subject, wherein at least 20% of the genes are selected from the brain tumor marker genes listed in Table 1.

3. A method of determining the prognosis of a subject with a brain tumor comprising measuring the level of at least one miRNA selected from Table 1 in a test biological sample from said subject, wherein the miRNA expression level is associated with an adverse prognosis; and an alteration in the level of the at least one miRNA in the test biological sample, relative to the level of a corresponding miRNA in a control biological sample, is indicative of an adverse prognosis.

4. The method of claim 1, wherein biological sample comprises a tissue sample containing cancer cells, biopsy fluid, blood or urine.

5. (canceled)

6. (canceled)

7. (canceled)

8. (canceled)

9. (canceled)

10. The method of claim 1, wherein providing a diagnosis comprises providing a providing a probability score.

11. (canceled)

12. The method of claim 1, wherein the plurality of brain tumor marker genes comprises at least three of the brain tumor marker genes listed in Table 1.

13. The method of claim 1, wherein the plurality of brain tumor marker genes comprises at least five of the brain tumor marker genes listed in Table 1.

14. The method of claim 1, wherein the plurality of brain tumor marker genes comprises at least ten of the brain tumor marker genes listed in Table 1.

15. (canceled)

16. (canceled)

17. The method of claim 1, wherein at least 30% of the genes are selected from the brain tumor marker genes listed in Table 1.

18. The method of claim 1, wherein at least 50% of the genes are selected from the brain tumor marker genes listed in Table 1.

19. The method of claim 1, wherein at least 75% of the genes are selected from the brain tumor marker genes listed in Table 1.

20. The method of claim 1, wherein at least 90% of the genes are selected from the brain tumor marker genes listed in Table 1.

21. (canceled)

22. A method of treating a brain tumor in a subject, comprising:
(a) determining the amount of at least one miRNA selected from the genes in Table 1 in cancer cells, relative to control cells; and
(b) altering the amount of miRNA expressed in the cells by
(i) administering to the subject an effective amount of at least one isolated miRNA, a precursor thereof, an isolated variant thereof, or a biologically active fragment thereof, or
(ii) administering to the subject an effective amount of at least one compound for inhibiting expression of the at least one miRNA.

23. (canceled)

24. The method of claim 21, wherein said brain tumor is glioblastoma multiforme.

25. (canceled)

26. The method of claim 22, wherein determining the amount of at least one miRNA selected from the genes in Table 1 in cancer cells, relative to control cells comprises (i) comparing (a) a set of expression profiles of brain tumor marker genes in a biological sample from the subject, the set comprising expression profiles of a plurality of brain tumor marker genes from Table 1, to (b) a set of expression profiles of brain tumor marker genes in a biological sample from a control subject.

27. The method of claim 26, further comprising obtaining the set of expression profiles prior to the comparing step.

28. The method of claim 22, wherein said cancer cells are obtained from a tissue sample containing cancer cells, biopsy fluid, blood or urine.

29. The method of claim 22, wherein at least 30% of the genes are selected from the brain tumor marker genes listed in Table 1.

30. The method of claim 22, wherein at least 50% of the genes are selected from the brain tumor marker genes listed in Table 1.

31. The method of claim 22, wherein at least 75% of the genes are selected from the brain tumor marker genes listed in Table 1.

32. The method of claim 22, wherein at least 90% of the genes are selected from the brain tumor marker genes listed in Table 1.

33. The method of claim 22, wherein said brain tumor is glioblastoma multiforme.
